(19) United States
(12) Patent Application Publication
(10) Pub. No.: US 2024/0140966 A1
(43) Pub. Date: May 2, 2024

(54) AMORPHOUS AND CRYSTALLINE FORMS OF MCL-1 ANTAGONISTS

(71) Applicant: AMGEN INC., Thousand Oaks, CA (US)

(72) Inventors: Yosup Rew, Thousand Oaks, CA (US); Van Luu, Thousand Oaks, CA (US); Manuel Zancanella, Thousand Oaks, CA (US); Mary Chaves, Arlington, MA (US)

(21) Appl. No.: 18/296,211

(22) Filed: Apr. 5, 2023

Related U.S. Application Data
(60) Provisional application No. 63/327,656, filed on Apr. 5, 2022.

(51) Int. Cl. C07D 513/10

(52) U.S. Cl. CPC C07D 513/10

(57) ABSTRACT

Disclosed herein are crystalline and amorphous forms of (1S,3′R,6′R,7′S,8′E,11′S,12′R)-6-chloro-7′-methoxy-11′,12′-dimethyl-3,4-dihydro-2H, 15′H-spiro[naphthalene-1,22′-[20]oxa[13]thia[1,14]diazatetracyclo[14.7.2.0^{3,6},0^{19,24}]pentacosa[8,16,18,24]tetraen]-15′-one 13′,13′-dioxide (AMG 176):

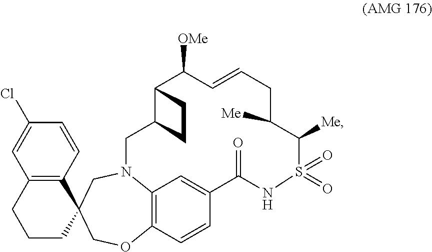

and salts and hydrates thereof. Also disclosed are methods of making the crystalline and amorphous forms, and methods of treating diseases and disorders with the crystalline and amorphous forms.

### BACKGROUND

### Technical Field

The present disclosure relates to crystalline and amorphous forms of (1S,3′R,6′R,7′S,8′E,11′S,12′R)-6-chloro-7′-methoxy-11′,12′-dimethyl-3,4-dihydro-2H,15′H-spiro[naphthalene-1,22′-[20]oxa[13]thia[1,14]diazatetracyclo[14.7.2.0^{3,6}.O^{19,24}]pentacosa[8,16,18,24]tetraen]-15′-one 13′,13′-dioxide (AMG 176), hydrates, and salts thereof, which functions as an inhibitor of myeloid cell leukemia 1 protein (Mcl-1).

### DESCRIPTION OF RELATED TECHNOLOGY

The compound, (1S,3′R,6′R,7′S,8 1E,11′S,12′R)-6-chloro-7′-methoxy-11′,12′-dimethyl-3,4-dihydro-2H,15H -spiro[naphthalene-1,22′-[20]oxa[13]thia[1, 14]diazatetracyclo[14.7.2.0^{3,6}.0 19,24]pentacosa[8,16,18,24]tetraen]-15′-one 13′,13′-dioxide (AMG 176), is useful as an inhibitor of myeloid cell leukemia 1 (“Mcl-1):

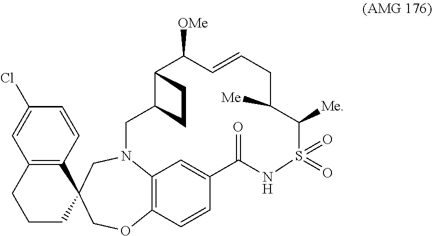

One common characteristic of human cancer is overexpression of Mcl-1. Mcl-1 overexpression prevents cancer cells from undergoing programmed cell death (apoptosis), allowing the cells to survive despite widespread genetic damage.

Mcl-1 is a member of the Bcl-2 family of proteins. The Bcl-2 family includes pro-apoptotic members (such as BAX and BAK) which, upon activation, form a homo-oligomer in the outer mitochondrial membrane that leads to pore formation and the escape of mitochondrial contents, a step in triggering apoptosis. Antiapoptotic members of the Bcl-2 family (such as Bcl-2, Bcl-XL, and Mcl-1) block the activity of BAX and BAK. Other proteins (such as BID, BIM, BIK, and BAD) exhibit additional regulatory functions. Research has shown that Mcl-1 inhibitors can be useful for the treatment of cancers. MCl-1 is overexpressed in numerous cancers.

U.S. Pat. No. 9,562,061, which is incorporated herein by reference in its entirety, discloses AMG 176 as an Mcl-1 inhibitor and provides a method for preparing it. However, alternative forms of AMG 176 with improved properties are desirable, particularly for clinical use of AMG 176.

### SUMMARY

Provided herein are crystalline and amorphous forms of AMG 176, wherein AMG 176 has the structure

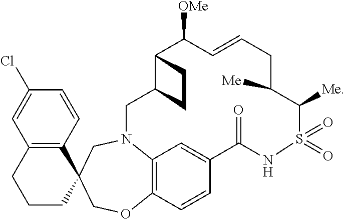

Also provided are crystalline forms of AMG 176, characterized by solid state ¹³C NMR peaks at 12.39, 19.40, 20.46, 27.24, 28.07, 30.54, 33.09, 33.80, 37.18, 41.80, 42.98, 54.86, 58.69, 60.01, 63.11, 80.06, 85.77, 117.20, 119.77, 120.86, 127.04, 129.01, 129.71, 131.30, 132.24, 133.58, 139.16, 140.11, 140.69, 152.08, and 169.82±0.5 ppm (“AMG 176 Form 1”).

Also provided are crystalline forms of AMG 176 characterized by XRPD pattern peaks at 13.0, 16.9, and 17.3±0.2° 2θ using Cu Kα radiation (“AMG 176 Form 2”).

Also provided are amorphous forms of AMG 176, having an XRPD pattern substantially as shown in FIG. 14 (“AMG 176 Amorphous”).

Also provided are crystalline forms of AMG 176, as a calcium salt hydrate, characterized by XRPD pattern peaks at 6.8, 7.8, and 15.5±0.2° 2θ0 using Cu Kα radiation (“AMG 176 Calcium Hydrate Form 1”).

Also provided are crystalline forms of AMG 176, as a calcium salt hydrate, characterized by XRPD pattern peaks at 6.2, 20.2, and 24.4±0.2° 2θ using Cu Kα radiation (“AMG 176 Calcium Hydrate Form 2”).

Also provided are crystalline forms of AMG 176, as a calcium salt hydrate, characterized by XRPD pattern peaks at 6.2, 6.7, and 8.2±0.2° 2θ using Cu Ka radiation (“AMG 176 Calcium Hydrate Form 3”).

Also provided are amorphous forms of AMG 176, as a calcium salt hydrate, having an XRPD pattern substantially as shown in FIG. 28 (“AMG 176 Amorphous Calcium Hydrate”).

Also provided are pharmaceutical formulations comprising the crystalline or amorphous forms of AMG 176 as described herein and a pharmaceutically acceptable excipient.

Also provided are methods of treating a subject suffering from cancer, comprising administering to the subject a therapeutically effective amount of the crystalline or amorphous forms of AMG 176 as described herein, or the pharmaceutical formulations comprising the crystalline or amorphous forms of AMG 176 as described herein and a pharmaceutically acceptable excipient.

### BRIEF DESCRIPTION OF THE DRAWINGS

FIG. 1 depicts a solid state 13 C NMR of the crystalline Form 1 of AMG 176.

FIG. 2 depicts an X-ray powder diffraction (“XRPD”) pattern of crystalline Form 1 of AMG 176.

FIG. 3 depicts a differential scanning calorimetry (“DSC”) thermograph of crystalline Form 1 of AMG 176.

FIG. 4 depicts a thermogravimetric analysis (“TGA”) trace of crystalline Form 1 of AMG 176.

FIG. 5 depicts a moisture sorption profile (DVS) of crystalline Form 1 of AMG 176.

FIG. 6 depicts a single crystal X-ray crystal structure of the unit cell of crystalline Form 1 of AMG 176.

FIG. 7 depicts an overlay of X-ray powder diffraction (“XRPD”) patterns of crystalline Form 1 of AMG 176 after solid state physical stability assessment. Form conversion was not observed after 8 weeks under stress conditions.

FIG. 8 depicts an X-ray powder diffraction (“XRPD”) pattern of the crystalline Form 2 of AMG 176.

FIG. 9 depicts a differential scanning calorimetry (“DSC”) thermograph of the crystalline Form 2 of AMG 176 indicating a onset and peak melting temperatures of approximately 233° C. and 238° C., respectively.

FIG. 10 depicts a differential scanning calorimetry (“DSC”) thermograph and thermogravimetric analysis (“TGA”) trace overlay of the crystalline 2-Me THF solvate Form 2 of AMG 176 showing a DSC onset at 176° C. and 12.8% TGA weight loss between 140-215° C.

FIG. 11 depicts a differential scanning calorimetry (“DSC”) thermograph and thermogravimetric analysis (“TGA”) trace overlay of the crystalline THF/water solvate Form 2 of AMG 176 showing a DSC onset at 177° C. and 8.9% TGA weight loss between 140-200° C.

FIG. 12 depicts a differential scanning calorimetry (“DSC”) thermograph and thermogravimetric analysis (“TGA”) trace overlay of the crystalline MTBE solvate Form 2 of AMG 176 showing a DSC onset at 163° C. and 12.7% TGA weight loss between 140-190° C.

FIG. 13 depicts a differential scanning calorimetry (“DSC”) thermograph and thermogravimetric analysis (“TGA”) trace overlay of the crystalline 1,4-dioxane/water solvate Form 2 of AMG 176 showing a DSC onset at 170° C. and 10.1% TGA weight loss between 120-180° C.

FIG. 14 depicts an X-ray powder diffraction (“XRPD”) pattern of the amorphous form of AMG 176.

FIG. 15 depicts a differential scanning calorimetry (“DSC”) thermograph of the amorphous form of AMG 176 indicating a Tm of approximately 163° C.

FIG. 16 depicts a thermogravimetric analysis (“TGA”) trace of the amorphous form of AMG 176 showing 5.4% weight loss to 200° C., with 11.6% weight loss to 275° C.

FIG. 17 depicts a thermogravimetric analysis (“TGA”) trace of the amorphous form of AMG 176 showing 3.8% weight loss to 180° C.

FIG. 18 depicts a moisture sorption profile (DVS) of the amorphous form of AMG 176 showing weight gain of about 0.95 wt % water vapor between 5% and 95% relative humidity. More weight was lost upon desorption (1.59%) than was gained on adsorption, likely due to the loss of residual DCM and/or water from the starting material. XRPD of the post-DVS material was consistent with amorphous material.

FIG. 19 depicts an overlay of XRPD analysis of amorphous AMG 176 samples after solid state physical stability assessment. Crystallization not observed after 8 weeks under stress conditions.

FIG. 20 depicts an X-ray powder diffraction (“XRPD”) pattern of the crystalline calcium salt hydrate Form 1 of AMG 176.

FIG. 21 depicts an overlaid differential scanning calorimetry (“DSC”) thermograph and thermogravimetric analysis (“TGA”) trace of the crystalline calcium salt hydrate Form 1 of AMG 176 indicating a melt at 301° C., recrystallization, and another melt at 322° C. followed by degradation, and about 5.5% TGA weight loss.

FIG. 22 depicts an X-ray powder diffraction (“XRPD”) pattern of the crystalline calcium salt hydrate Form 2 of AMG 176.

FIG. 23 depicts a moisture sorption profile (DVS) of the crystalline calcium salt hydrate Form 2 of AMG 176 showing weight gain of about 5.2% at 70% relative humidity and 23% weight gain at 95% relative humidity, which is likely due to the presence of residual NaCl.

FIG. 24 depicts an X-ray powder diffraction (“XRPD”) pattern of the crystalline calcium salt hydrate form 3 of AMG 176.

FIG. 25 depicts a differential scanning calorimetry (“DSC”) thermograph of the crystalline calcium salt hydrate form 3 of AMG 176 indicating a Tm of 314° C.

FIG. 26 depicts a thermogravimetric analysis (“TGA”) trace of the crystalline calcium salt hydrate form 3 of AMG 176 showing 3.5% weight loss.

FIG. 27 depicts a moisture sorption profile (DVS) of the crystalline calcium salt hydrate form 3 of AMG 176 showing weight gain of about 7.4% of moisture at 95% relative humidity.

FIG. 28 depicts an X-ray powder diffraction (“XRPD”) pattern of the amorphous calcium salt form of AMG 176.

FIG. 29 depicts an overlay of differential scanning calorimetry (“DSC”) thermographs of forms of the calcium salt of AMG 176 (top to bottom: crystalline Form 2, amorphous form, and crystalline Form 1) indicating a Tm of 323° C., 295° C., and 322° C., respectively.

FIG. 30 depicts an overlay of thermogravimetric analysis (“TGA”) traces of forms of the calcium salt of AMG 176 (top to bottom: amorphous form, crystalline Form 2, and crystalline Form 1) showing about 2.0% weight loss, about 3.1% weight loss, and about 5.5% weight loss, respectively.

FIG. 31 depicts a moisture sorption profile (DVS) of the amorphous calcium salt form of AMG 176 showing weight gain of about 9% to 95% relative humidity.

### DETAILED DESCRIPTION

AMG 176 is a small molecule targeting the Mc1-1 pathway for the treatment of hematologic malignancies. High throughput (HTS) and manual screening experiments conducted to date were unsuccessful in isolating and/or scaling up an acceptable salt or cocrystal form for development. Polymorph screening on the free acid identified polymorphs disclosed herein, such as the crystalline free acid, AMG 176 Form 1, as a suitable form for development to support intravenous (IV) drug product development. AMG 176 polymorphs disclosed herein can also be amendable for oral delivery as an amorphous solid dispersion, if desired.

Disclosed herein are crystalline and amorphous forms of ((1S,3′R,6′R,7′S,8′E,11′S,12′R)-6-chloro-740 -methoxy-11′,12′-dimethyl-3,4-dihydro-2H,15′H-spiro[naphthalene-1,22′-[20]oxa[13]thia[1,14]diazatetracyclo[14.7.2.0^{3,6}.0^{19,24]}pentacosa[8,16,18,24]tetraen]-15′-one 13′,13′-dioxide (AMG 176), hydrates, and salts thereof:

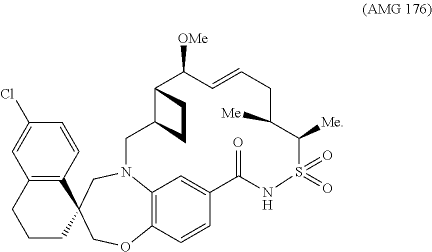

AMG 176 crystalline form 1 is an anhydrous/non-solvated AMG 176 form and is likely thermodynamically stable between 2 and 79° C. AMG 176 crystalline form 1, can be advantageous over other, solvated forms due to improved properties for drug formulation.

Also provided herein are pharmaceutical formulations of crystalline and amorphous forms of AMG 176, and methods of treating a subject suffering from cancer, comprising administering to the subject a therapeutically effective amount of a pharmaceutical formulation of a crystalline or amorphous form as disclosed herein.

U.S. Pat. No. 9,562,061, which is incorporated by reference herein in its entirety, discloses synthetic procedures for synthesizing Mcl-1 inhibitors, such as AMG 176.

Further provided herein are crystalline hydrate forms of AMG 176, pharmaceutical formulations thereof, and methods of treating a subject suffering from cancer, comprising administering to the subject a therapeutically effective amount of a pharmaceutical formulation of a crystalline hydrate form as disclosed herein.

The compounds disclosed herein may be identified either by their chemical structure and/or chemical name herein. When the chemical structure and chemical name conflict, the chemical structure is determinative of the identity of the compound.

When ranges are used herein for physical properties, such as molecular weight, or chemical properties, such as chemical formulae, all combinations and subcombinations of ranges and specific embodiments therein are intended to be included.

As used herein, chemical structures which contain one or more stereocenters depicted with dashed and bold bonds (i.e., [symbol] and [symbol] ) are meant to indicate absolute stereochemistry of the stereocenter(s) present in the chemical structure. As used herein, bonds symbolized by a simple line do not indicate a stereo-preference. Unless otherwise indicated to the contrary, chemical structures that include one or more stereocenters which are illustrated herein without indicating absolute or relative stereochemistry encompass all possible stereoisomeric forms of the compound (e.g., diastereomers, enantiomers) and mixtures thereof. Structures with a single bold or dashed line, and at least one additional simple line, encompass a single enantiomeric series of all possible diastereomers.

The term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

“Treatment” or “treating” means any treatment of a disease in a patient, including: a) preventing the disease, that is, causing the clinical symptoms of the disease not to develop; b) inhibiting the disease; c) slowing or arresting the development of clinical symptoms; and/or d) relieving the disease, that is, causing the regression of clinical symptoms. Treatment of diseases and disorders herein is intended to also include the prophylactic administration of a pharmaceutical formulation described herein to a subject (i.e., an animal, preferably a mammal, most preferably a human) believed to be in need of treatment, such as, for example, cancer.

The term “therapeutically effective amount” means an amount effective, when administered to a human or non-human patient, to treat a disease, e.g., a therapeutically effective amount may be an amount sufficient to treat a disease or disorder responsive to inhibition of Mcl-1. The therapeutically effective amount may be ascertained experimentally, for example by assaying blood concentration of the chemical entity, or theoretically, by calculating bioavailability.

The term “solvate” refers to the chemical entity formed by the interaction of a solvate and a compound. Crystalline solvates of AMG 176 used in formulations herein are specifically contemplated. Solvents that can form crystalline solvate forms of AMG 176 include without limitation, 2-methyltetrahydrofuran (2-Me THF), tetrahydrofuran (THF), methyl tert-butyl ether (MTBE), 1,4-dioxane, water, and combinations thereof. In some cases, a solvate has 0.5 to 2 solvent molecules per AMG 176 molecule.

The term “hydrate” is a specific type of solvate, where the solvent is water. A hydrate, as used herein, can have a variable amount of water including, e.g., hemi-hydrates, monohydrates, dihydrates, trihydrates, etc. Crystalline hydrates of AMG 176 are specifically contemplated for use in the formulations disclosed herein. In some cases, the hydrate has 0.5 to 2 water molecules pe AMG 176 molecule.

The term “polymorph” as used herein includes all crystalline and amorphous forms of the compound, including, for example, polymorphs, pseudopolymorphs, solvates, hydrates, unsolvated polymorphs (including anhydrates), and conformational polymorphs, as well as mixtures thereof, unless a particular crystalline or amorphous form is referred to. In some embodiments, the disclosure provides crystalline forms of AMG 176, for example, crystalline polymorphs, pseudopolymorphs, solvates, hydrates, unsolvated polymorphs (including anhydrates), and conformational polymorphs, as well as mixtures thereof, unless a particular crystalline form is referred to.

### Crystalline Forms

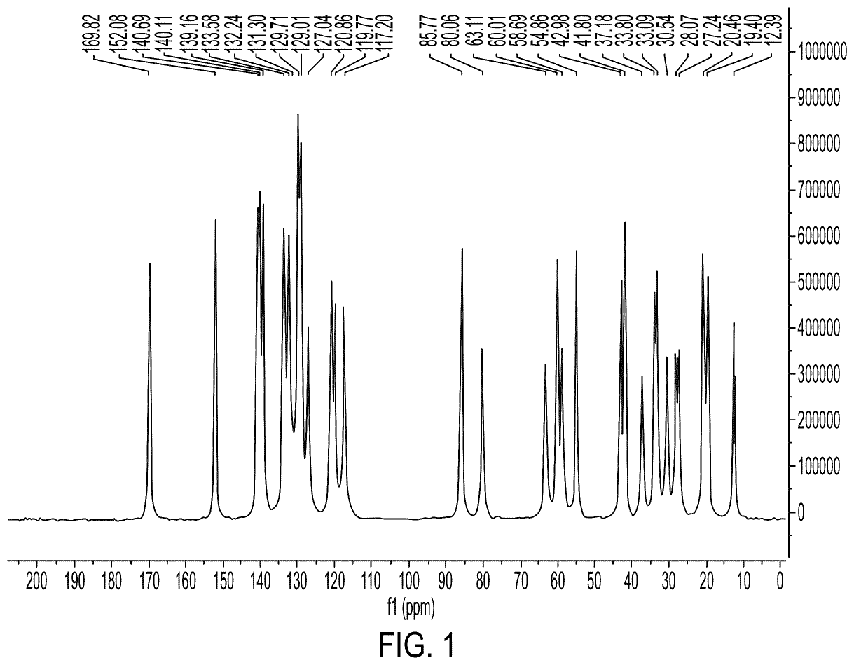

Crystalline Form 1: The AMG 176 crystalline Form 1 can be characterized by solid state 13 C NMR, obtained as set forth in the Examples, having peaks at 12.39, 19.40, 20.46, 27.24, 28.07, 30.54, 33.09, 33.80, 37.18, 41.80, 42.98, 54.86, 58.69, 60.01, 63.11, 80.06, 85.77, 117.20, 119.77, 120.86, 127.04, 129.01, 129.71, 131.30, 132.24, 133.58, 139.16, 140.11, 140.69, 152.08, and 169.82±0.5 ppm. In some embodiments, AMG 176 form 1 has a solid state ¹³C NMR substantially as shown in FIG. 1, wherein by “substantially” is meant that the reported peaks can vary by ±0.5 ppm.

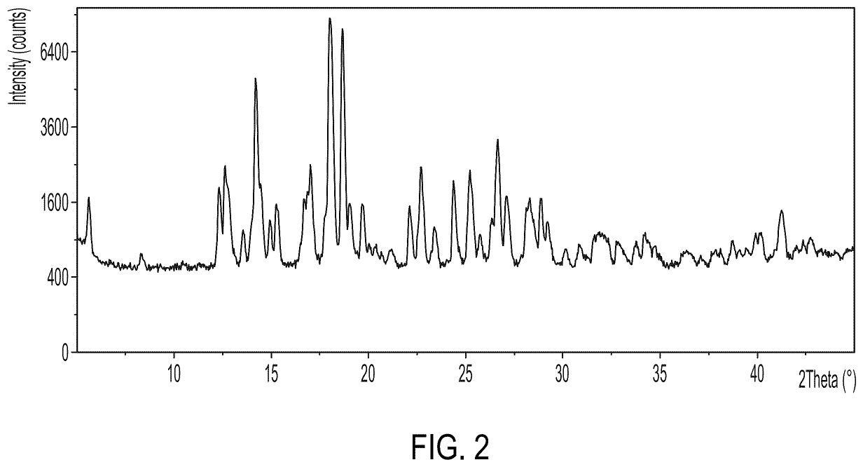

AMG 176 Form 1 can be characterized by an X-ray powder diffraction pattern, obtained as set forth in the Examples, having peaks at 14.2, 18.0, and 18.6±0.2° 2θ using Cu Kα radiation, optionally further characterized by additional peaks at 12.6, 17.0, 22.7, and 26.6±0.2° 2θ using Cu Kα radiation, and/or additional peaks at 5.6, 12.3, 12.8, 14.5, 16.7, 24.4, 25.2, 27.1, 28.3, and 28.9±0.2° 2θ using Cu Kα radiation. In some embodiments, AMG 176 Form 1 has an X-ray powder diffraction pattern substantially as shown in FIG. 2, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

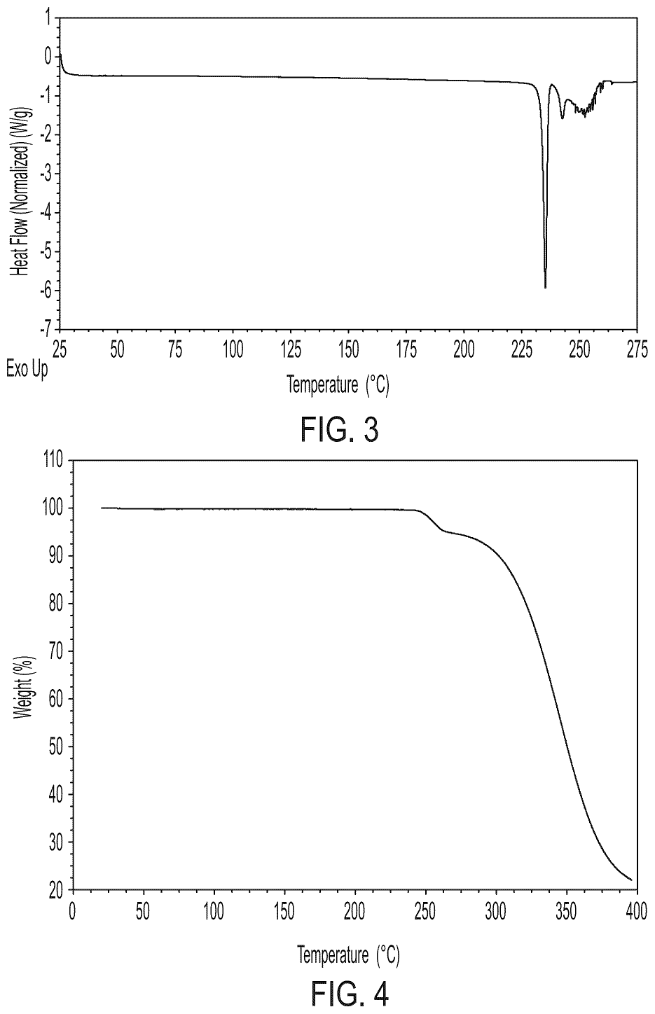

Differential scanning calorimetry (DSC) thermographs were obtained, as set forth in the Examples, for AMG 176 Form 1. The DSC curve indicates an endothermic transition at 236° C.±3° C. Thus, in some embodiments, the amorphous form of AMG 176 Form 1 can be characterized by a DSC thermograph having a transition endotherm with an onset of about 234° C. For example, in some embodiments AMG 176 Form 1 is characterized by DSC, as shown in FIG. 3.

AMG 176 Form 1 can be characterized by thermogravimetric analysis (TGA). Thus, AMG 176 Form 1 can be characterized by a weight loss in a range of about 5.3% with an onset temperature of about 234° C. In some embodiments, AMG 176 Form 1 has a thermogravimetric analysis substantially as depicted in FIG. 4, wherein by “substantially” is meant that the reported TGA features can vary by ±5° C.

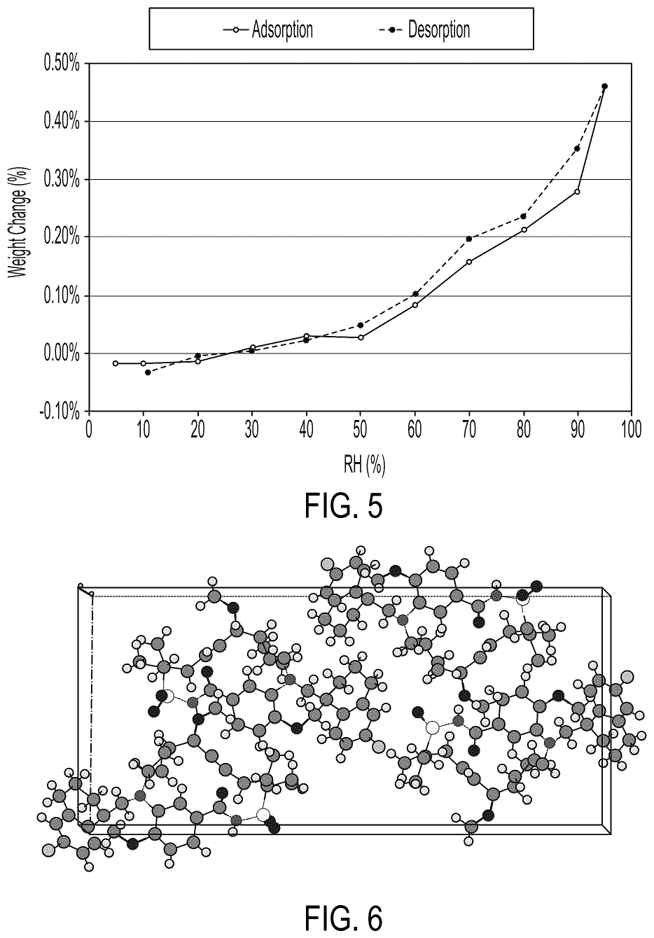

AMG 176 Form 1 can be characterized by a moisture sorption profile. For example, in some embodiments, AMG 176 Form 1 is characterized by the moisture sorption profile as shown in FIG. 5, showing that AMG 176 Form 1 is non-hygroscopic up to 95% RH.

AMG 176 Form 1 can be characterized by a single crystal structure substantially as shown in FIG. 6, or as set forth in the Examples.

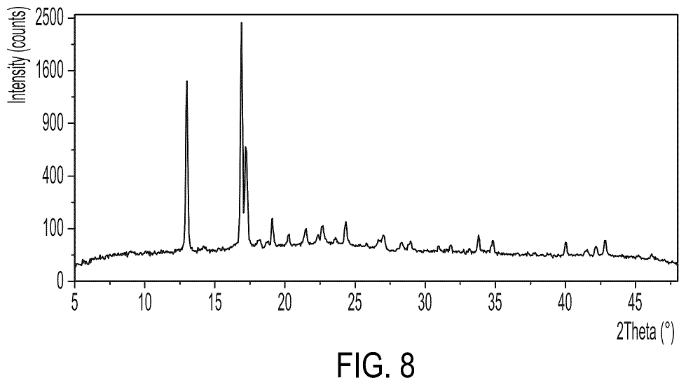

Crystalline Form 2: AMG 176 crystalline Form 2 can be characterized by an X-ray powder diffraction pattern, obtained as set forth in the Examples, having peaks at 13.0, 16.9, and 17.3±0.2° 2θ using Cu Kα radiation, optionally further characterized by additional peaks at 19.1, 21.5, 22.7, 24.3, 27.0, 33.8, 40.0, and 42.8±0.2° 2θ using Cu Kα radiation. In some embodiments, AMG 176 Form 2 has an X-ray powder diffraction pattern substantially as shown in FIG. 8, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

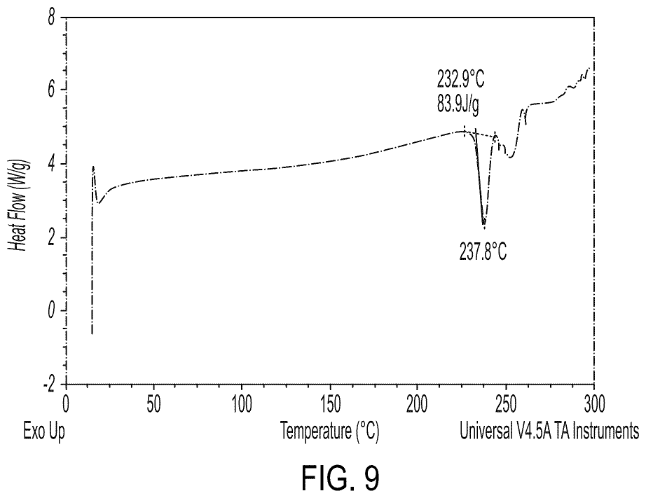

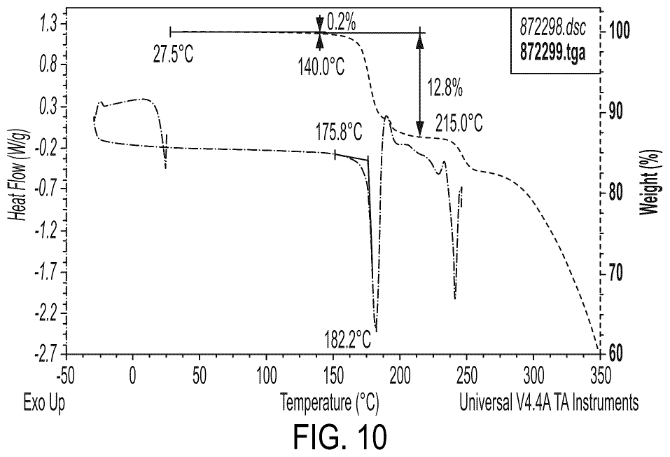

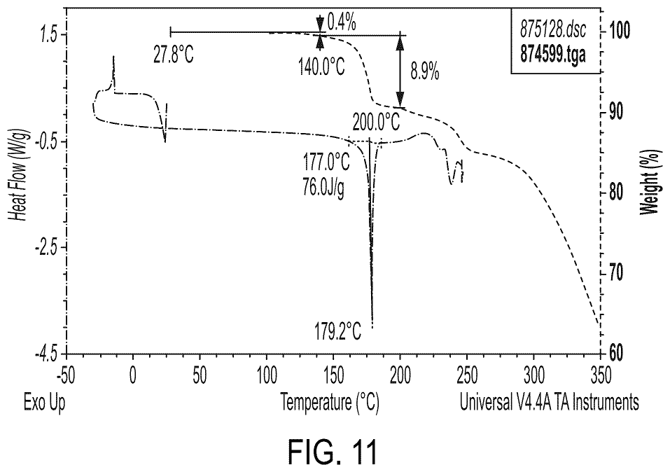

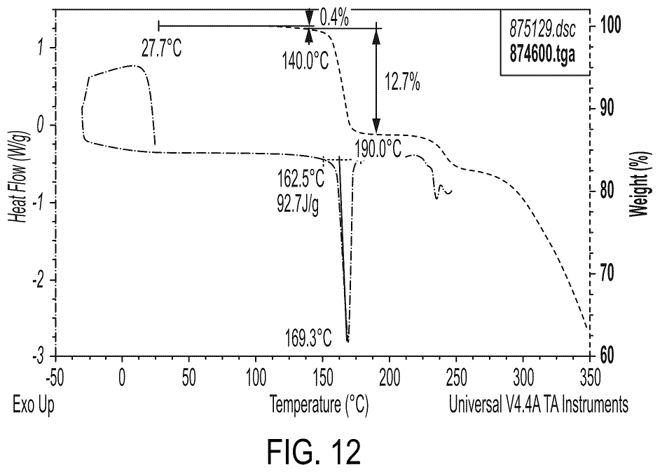

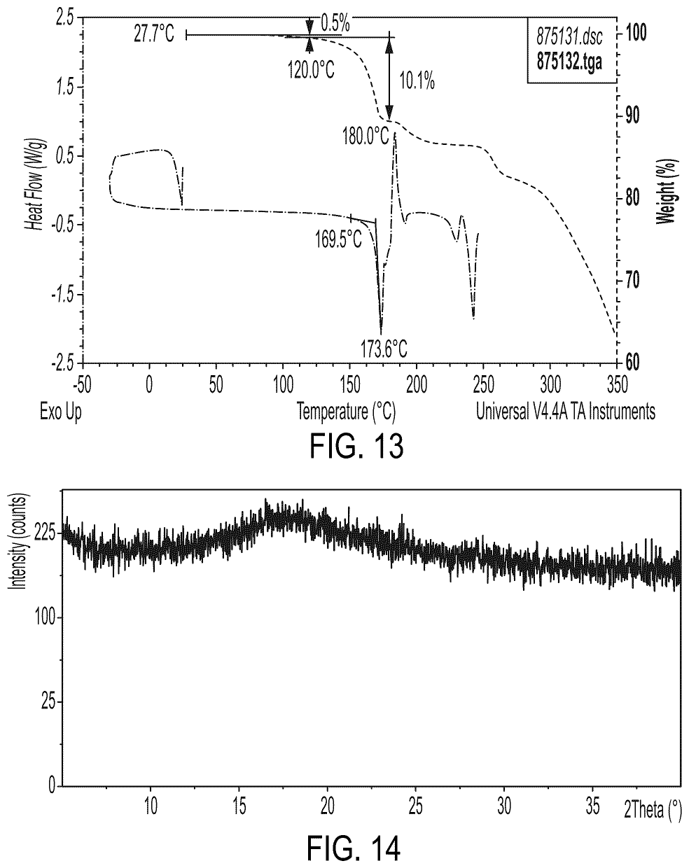

Differential scanning calorimetry (DSC) thermographs were obtained, as set forth in the Examples, for AMG 176 Form 2. The DSC curve indicates an endothermic transition with an onset of 162° C. to 169° C., 169° C. to 173° C., 177° C. to 179° C., 178° C. to 182° C., or 232° C. to 238° C. Thus, in some embodiments, AMG 176 Form 2 can be characterized by a DSC thermograph having a transition endotherm with an onset of about 163° C.±3° C., 170° C.±3° C., 176° C.±3° C., 177° C.±3° C., or 238° C.±3° C. For example, in some embodiments AMG 176 Form 2 is characterized by DSC, as shown in FIG. 9, FIG. 10, FIG. 11, FIG. 12, or FIG. 13. In some embodiments, the DSC curve indicates an endothermic transition with an onset of 232° C. to 238° C., i.e., AMG 176 Form 2 can be characterized by a DSC thermograph having a transition endotherm with an onset of about 238° C. ±3° C. For example, in some embodiments AMG 176 Form 2 is characterized by DSC, as shown in FIG. 9. In some embodiments, the DSC curve indicates an endothermic transition with an onset of 177° C. to 179° C., i.e., AMG 176 Form 2 can be characterized by a DSC thermograph having a transition endotherm with an onset of about 176° C.±3° C. For example, in some embodiments AMG 176 Form 2 is characterized by DSC, as shown in FIG. 10. In some embodiments, the DSC curve indicates an endothermic transition with an onset of 178° C. to 182° C., i.e., AMG 176 Form 2 can be characterized by a DSC thermograph having a transition endotherm with an onset of about 177° C.±3° C. For example, in some embodiments AMG 176 Form 2 is characterized by DSC, as shown in FIG. 11. In some embodiments, the DSC curve indicates an endothermic transition with an onset of 162° C. to 169° C., i.e., AMG 176 Form 2 can be characterized by a DSC thermograph having a transition endotherm with an onset of about 163° C.±3° C. In some embodiments AMG 176 Form 2 is characterized by DSC, as shown in FIG. 12. In some embodiments, the DSC curve indicates an endothermic transition with an onset of 169° C. to 173° C., i.e., AMG 176 Form 2 can be characterized by a DSC thermograph having a transition endotherm with an onset of about 170° C.±3° C. In some embodiments AMG 176 Form 2 is characterized by DSC, as shown in FIG. 13.

AMG 176 Form 2 can be characterized by thermogravimetric analysis (TGA). Thus, AMG 176 Form 2 can be characterized by a weight loss in a range of about 12.8% between 140-215° C., about 8.9% between 140-200° C., about 12.7% between 140-190° C., or about 10.1% between 120-180° C. In some embodiments, AMG 176 Form 2 can be characterized by a weight loss in a range of about 12.8% between 140-215° C. In some embodiments, AMG 176 Form 2 can be characterized by a weight loss in a range of about about 8.9% between 140-200° C. In some embodiments, AMG 176 Form 2 can be characterized by a weight loss in a range of about about 12.7% between 140-190° C. In some embodiments, AMG 176 Form 2 can be characterized by a weight loss in a range of about 10.1% between 120-180° C. In some embodiments, AMG 176 Form 1 has a thermogravimetric analysis substantially as depicted in FIG. 10, FIG. 11, FIG. 12, or FIG. 13, wherein by “substantially” is meant that the reported TGA features can vary by ±5° C. In some embodiments, AMG 176 Form 1 has a thermogravimetric analysis substantially as depicted in FIG. 10. In some embodiments, AMG 176 Form 1 has a thermogravimetric analysis substantially as depicted in FIG. 11. In some embodiments, AMG 176 Form 1 has a thermogravimetric analysis substantially as depicted in FIG. 12. In some embodiments, AMG 176 Form 1 has a thermogravimetric analysis substantially as depicted in FIG. 13.

Amorphous AMG 176 can be characterized by an X-ray powder diffraction pattern, obtained as set forth in the Examples. In some embodiments, amorphous AMG 176 has an X-ray powder diffraction pattern substantially as shown in FIG. 14, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

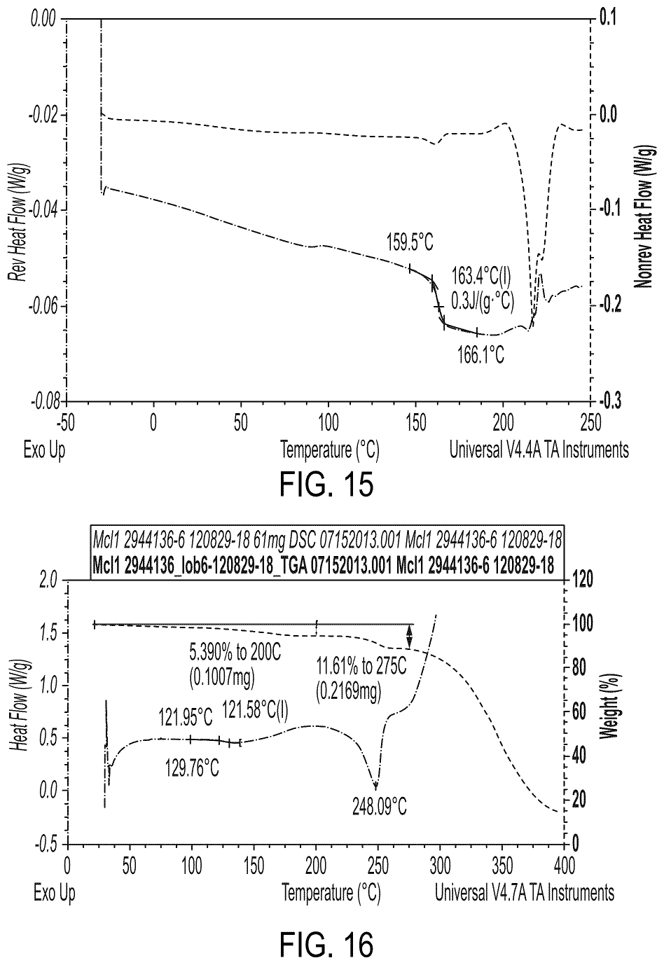

Differential scanning calorimetry (DSC) thermographs were obtained, as set forth in the Examples, for amorphous AMG 176. The DSC curve indicates an endothermic transition at 122° C.±3° C. or 163° C.±3° C. In some embodiments, the DSC curve indicates an endothermic transition at 122° C. ±3° C. In some embodiments, the DSC curve indicates an endothermic transition at 163° C. ±3° C. Thus, in some embodiments, amorphous AMG 176 can be characterized by a DSC thermograph having a transition endotherm with an onset of 122° C. to 130° C. or 159° C. to 166° C. In some embodiments, amorphous AMG 176 can be characterized by a DSC thermograph having a transition endotherm with an onset of 122° C. to 130° C. In some embodiments, amorphous AMG 176 can be characterized by a DSC thermograph having a transition endotherm with an onset of 159° C. to 166° C. For example, in some embodiments amorphous AMG 176 is characterized by DSC, as shown in FIG. 15 or FIG. 16. In some embodiments amorphous AMG 176 is characterized by DSC as shown in FIG. 15. In some embodiments amorphous AMG 176 is characterized by DSC as shown in FIG. 16.

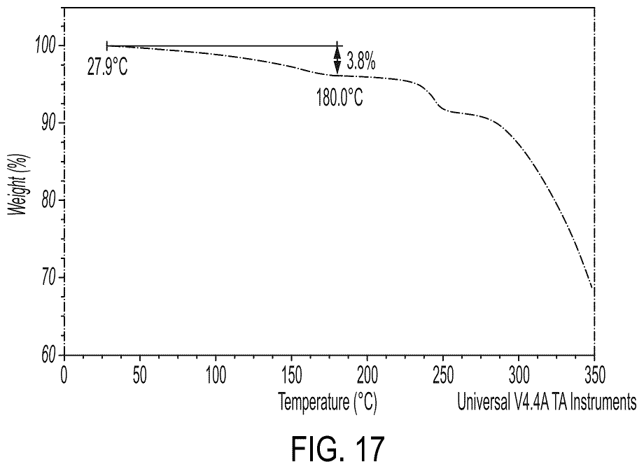

Amorphous AMG 176 can be characterized by thermogravimetric analysis (TGA). Thus, amorphous AMG 176 can be characterized by a weight loss of about 3.8 wt % between 28 and 180° C. In some embodiments, anhydrous AMG 176 has a thermogravimetric analysis substantially as depicted in FIG. 17, wherein by “substantially” is meant that the reported TGA features can vary by ±5° C.

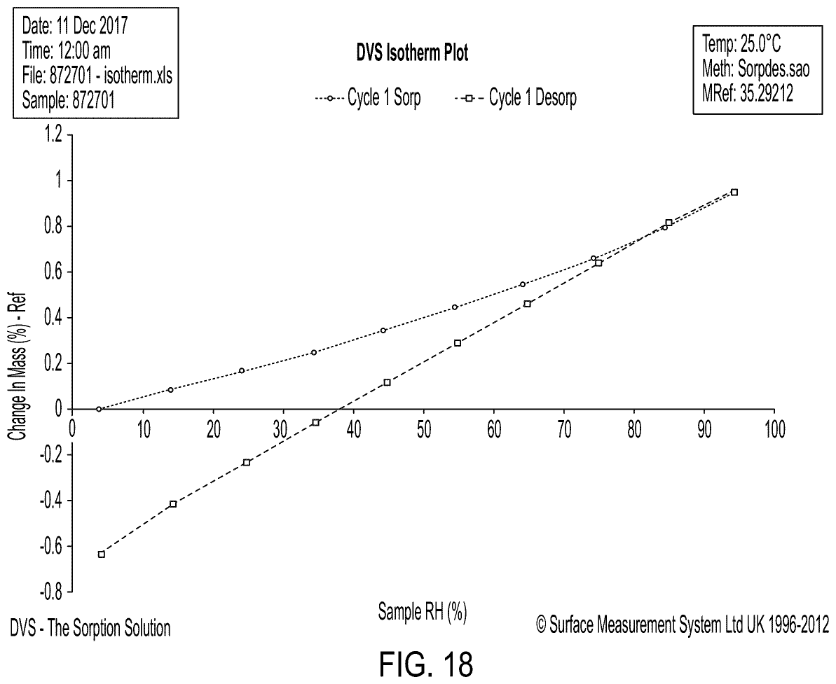

Amorphous AMG 176 can be characterized by a moisture sorption profile. For example, in some embodiments, amorphous AMG 176 is characterized by the moisture sorption profile as shown in FIG. 18, showing a weight gain of 0.95% by 95% RH.

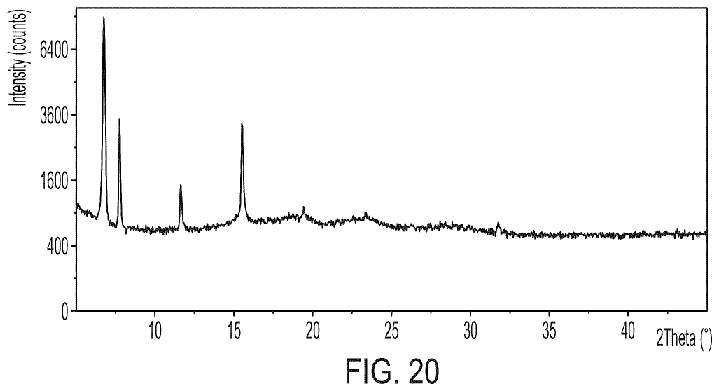

AMG 176 Calcium Salt Hydrate Form 1 can be characterized by an X-ray powder diffraction pattern, obtained as set forth in the Examples, having peaks at 6.8, 7.8, and 15.5±0.2°20 αusing Cu Kα radiation, optionally further characterized by additional peaks at 11.6, 19.4, and 31.8±0.2° 2α using Cu Kα radiation. In some embodiments, AMG 176 Calcium Salt Hydrate Form 1 has an X-ray powder diffraction pattern substantially as shown in FIG. 20, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

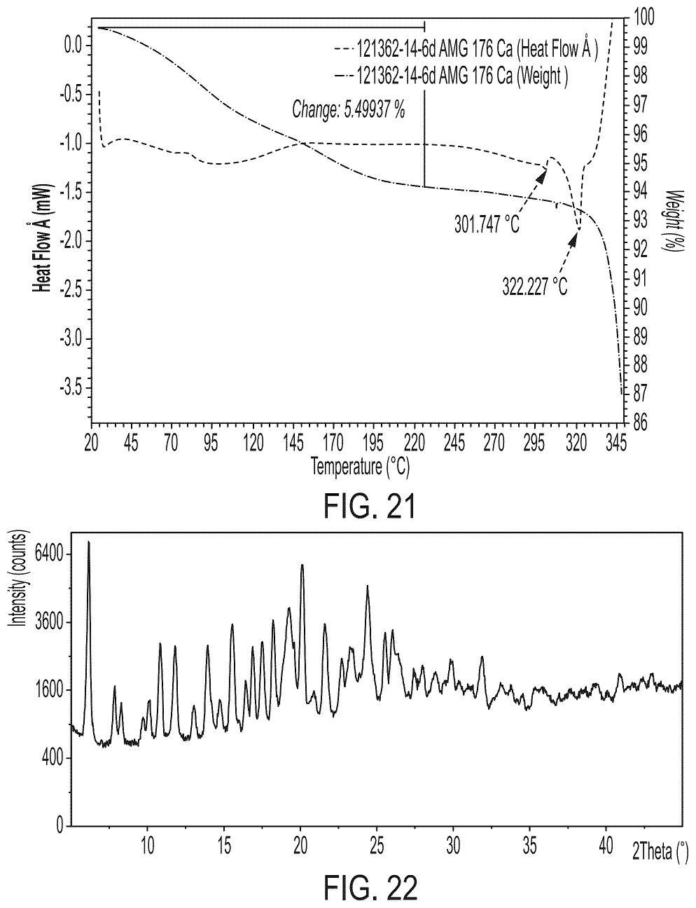

Differential scanning calorimetry (DSC) thermographs were obtained, as set forth in the Examples, for AMG 176 Calcium Salt Hydrate Form 1. The DSC curve indicates an endothermic transition at endothermic transitions at 301° C.±3° C. and 322° C.±3° C. Thus, in some embodiments, AMG 176 Calcium Salt Hydrate Form 1 can be characterized by a DSC thermograph having a transition endotherm with onsets of 298° C. to 304° C. and 319° C. to 325° C. For example, in some embodiments AMG 176 Calcium Salt Hydrate Form 1 is characterized by DSC, as shown in FIG. 21.

AMG 176 Calcium Salt Hydrate Form 1 can be characterized by thermogravimetric analysis (TGA). Thus, AMG 176 Calcium Salt Hydrate Form 1 can be characterized by a weight loss of about 5.5% weight loss that was confirmed to be due to the loss of water and ethanol by TGA-IR. In some embodiments, AMG 176 Calcium Salt Hydrate Form 1 has a thermogravimetric analysis substantially as depicted in FIG. 21, wherein by “substantially” is meant that the reported TGA features can vary by ±5° C.

AMG 176 Calcium Salt Hydrate Form 2 can be characterized by an X-ray powder diffraction pattern, obtained as set forth in the Examples, having peaks at 6.2, 20.2, and 24.4±0.2° 2θ using Cu Kα radiation, optionally further characterized by additional peaks at 15.5, 18.2, 19.3, and 21.6±0.2° 2θ using Cu Kα radiation, and/or additional peaks at 10.8, 11.8, 13.9, 16.9, 17.5, 19.6, 22.7, 23.3, 23.5, 25.5, 26.0, and 26.5±0.2° 2θ using Cu Kα radiation. In some embodiments, AMG 176 Calcium Salt Hydrate Form 2 has an X-ray powder diffraction pattern substantially as shown in FIG. 22, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

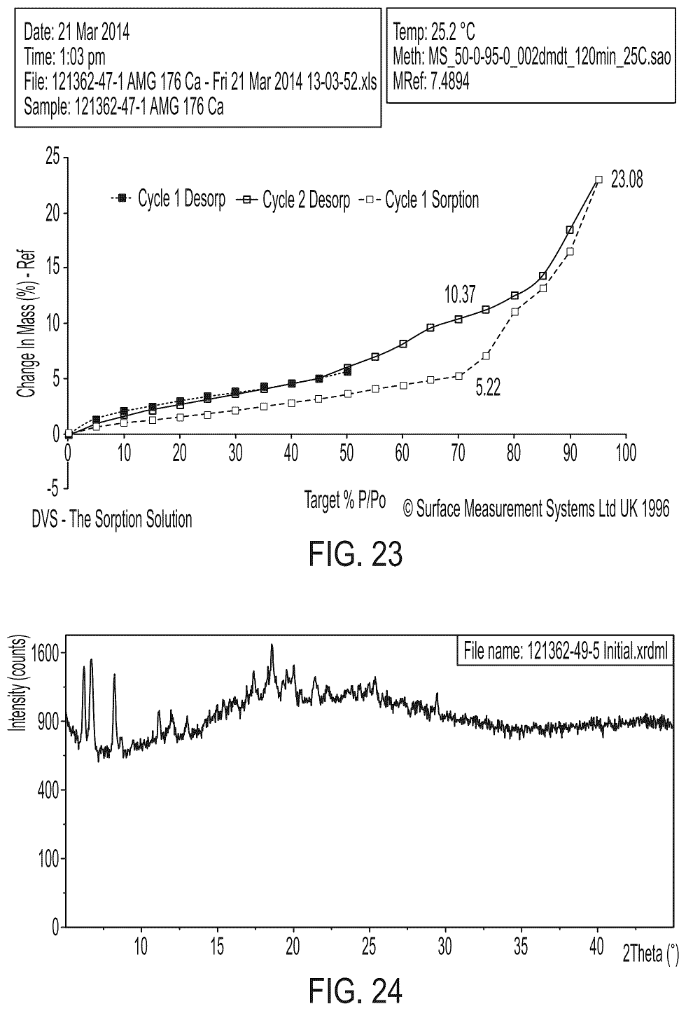

AMG 176 Calcium Salt Hydrate Form 2 can be characterized by a moisture sorption profile. For example, in some embodiments, AMG 176 Calcium Salt Hydrate Form 2 is characterized by the moisture sorption profile as shown in FIG. 23, showing a weight gain of approximately 5.2% at 70% RH and 23% weight gain at 95% RH.

AMG 176 Calcium Salt Hydrate Form 3 can be characterized by an X-ray powder diffraction pattern, obtained as set forth in the Examples, having peaks at 6.2, 6.7, and 8.2±0.2° 2 θ using Cu Kα0 radiation, optionally further characterized by additional peaks at 17.4, 18.6, and 20.0±0.2° 2θ using Cu Kα radiation, and/or additional peaks at 11.1, 12.0, 13.0, 21.4, 25.4, and 29.4±0.2° 2θ using Cu Kα radiation. In some embodiments, AMG 176 Calcium Salt Hydrate Form 3 has an X-ray powder diffraction pattern substantially as shown in FIG. 24, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

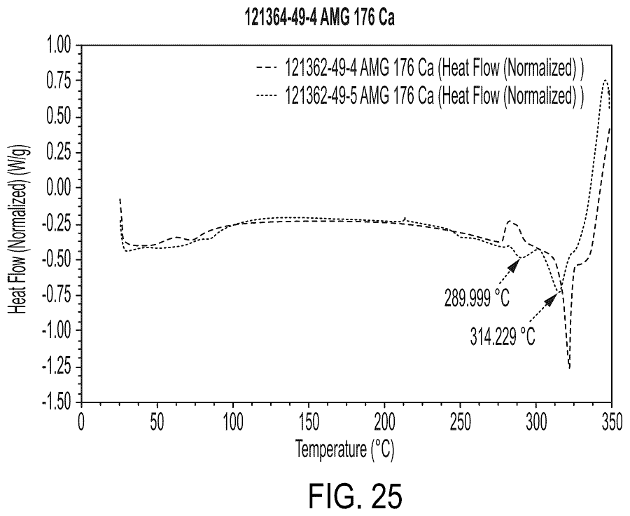

Differential scanning calorimetry (DSC) thermographs were obtained, as set forth in the Examples, for AMG 176 Calcium Salt Hydrate Form 3. The DSC curve indicates an endothermic transition at 314° C.±3° C. Thus, in some embodiments, AMG 176 Calcium Salt Hydrate Form 3 can be characterized by a DSC thermograph having a transition endotherm with an onset of 311° C. to 317° C. For example, in some embodiments AMG 176 Calcium Salt Hydrate Form 3 is characterized by DSC, as shown in FIG. 25.

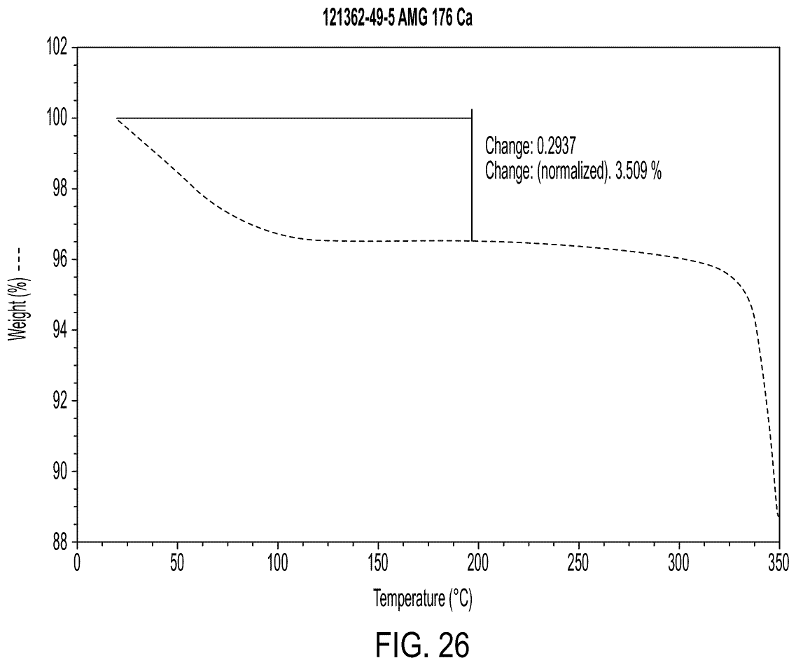

AMG 176 Calcium Salt Hydrate Form 3 can be characterized by thermogravimetric analysis (TGA). Thus, AMG 176 Calcium Salt Hydrate Form 3 can be characterized by a weight loss of about 3.5%. In some embodiments AMG 176 Calcium Salt Hydrate Form 3 has a thermogravimetric analysis substantially as depicted in FIG. 26, wherein by “substantially” is meant that the reported TGA features can vary by ±5° C.

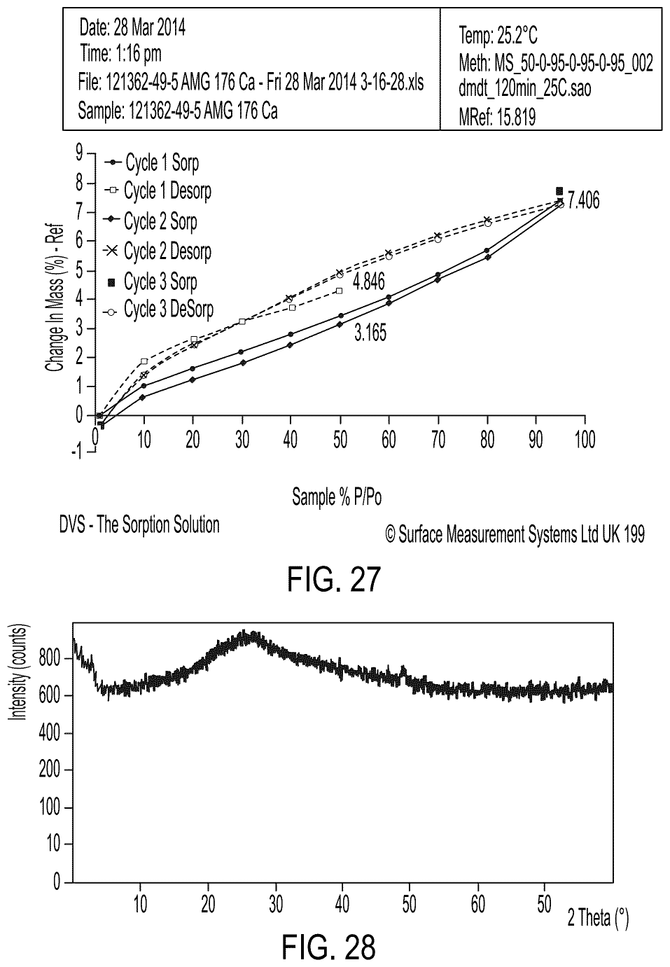

AMG 176 Calcium Salt Hydrate Form 3 can be characterized by a moisture sorption profile. For example, in some embodiments, AMG 176 Calcium Salt Hydrate Form 3 is characterized by the moisture sorption profile as shown in FIG. 27, showing a weight gain of 7.4% by 95% RH.

Amorphous AMG 176 Calcium Salt Hydrate can be characterized by an X-ray powder diffraction pattern, obtained as set forth in the Examples, substantially as shown in FIG. 28, wherein by “substantially” is meant that the reported peaks can vary by ±0.2°. It is well known in the field of XRPD that while relative peak heights in spectra are dependent on a number of factors, such as sample preparation and instrument geometry, peak positions are relatively insensitive to experimental details.

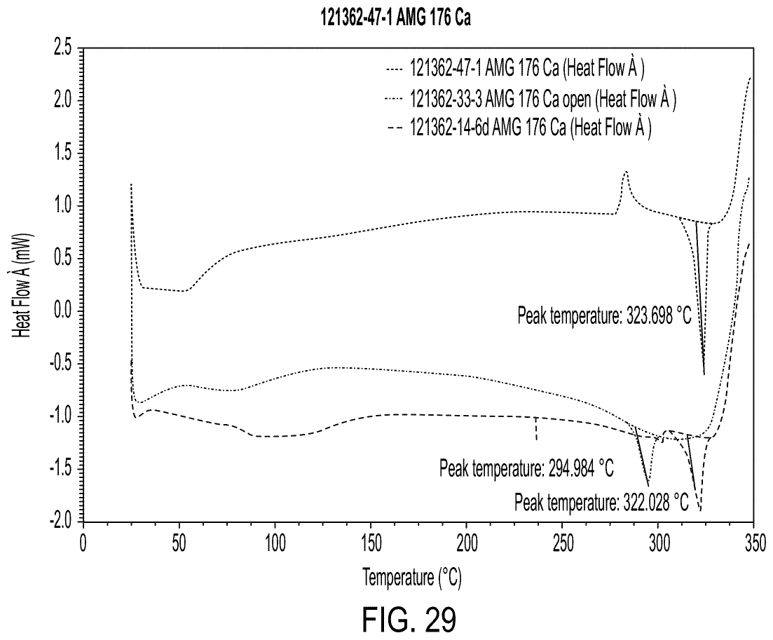

Differential scanning calorimetry (DSC) thermographs were obtained, as set forth in the Examples, for Amorphous AMG 176 Calcium Salt Hydrate. The DSC curve indicates an endothermic transition at 292° C.±3° C. Thus, in some embodiments, Amorphous AMG 176 Calcium Salt Hydrate can be characterized by a DSC thermograph having a transition endotherm with an onset of 289° C. to 295° C. For example, in some embodiments Amorphous AMG 176 Calcium Salt Hydrate is characterized by DSC, as shown in FIG. 29.

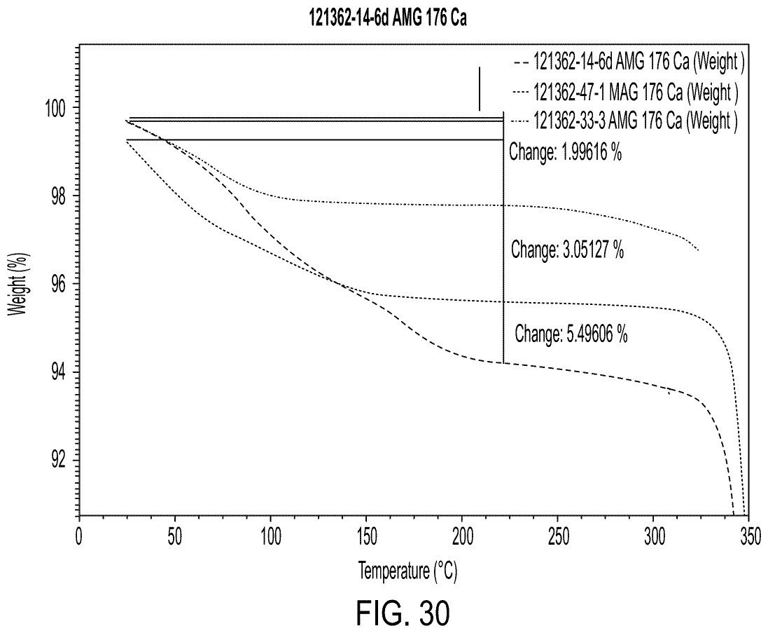

Amorphous AMG 176 Calcium Salt Hydrate can be characterized by thermogravimetric analysis (TGA). Thus, Amorphous AMG 176 Calcium Salt Hydrate can be characterized by a weight loss in a range of about 2%. In some embodiments, Amorphous AMG 176 Calcium Salt Hydrate has a thermogravimetric analysis substantially as depicted in FIG. 30, wherein by “substantially” is meant that the reported TGA features can vary by ±5° C.

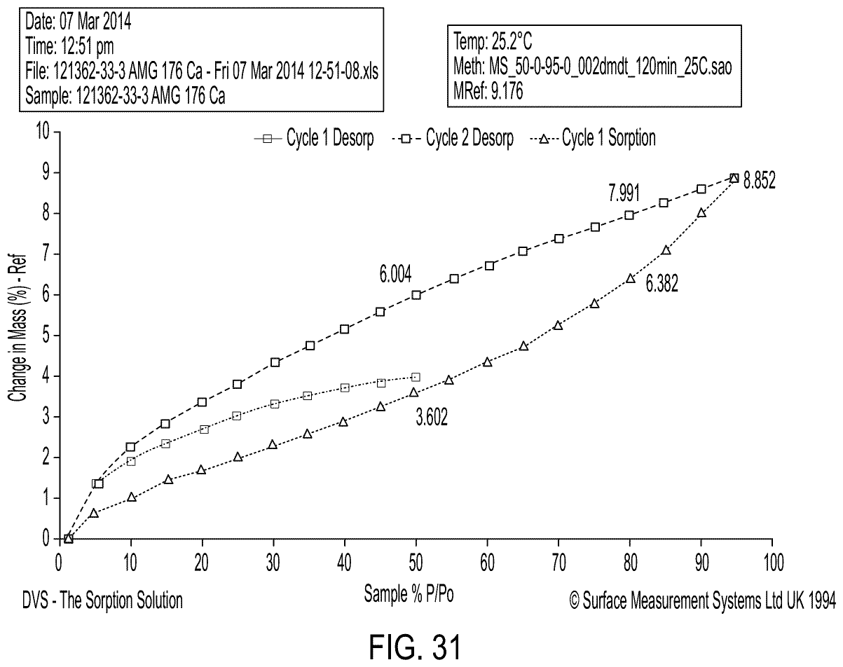

Amorphous AMG 176 Calcium Salt Hydrate can be characterized by a moisture sorption profile. For example, in some embodiments, Amorphous AMG 176 Calcium Salt Hydrate is characterized by the moisture sorption profile as shown in FIG. 31, showing a weight gain of approximately 9% by 95% RH.

### Pharmaceutical Formulations

Provided herein are pharmaceutical formulations comprising a crystalline form as disclosed herein and a pharmaceutically acceptable excipient.

In some embodiments, the pharmaceutical formulation is in the form of a tablet. In some embodiments, the pharmaceutical formulation is in the form of an immediate release tablet. Solid oral drug compositions (e.g., tablets) or preparations have various release profiles, such as an immediate release profile as referenced by FDA guidelines (“Dissolution Testing of Immediate Release Solid Oral Dosage Forms”, issued August 1997, Section IV-A). In the dissolution testing guideline for immediate release profiles, materials which dissolve at least 80% in the first 30 to 60 minutes in solution qualify as immediate release profiles. Therefore, immediate release solid dosage forms permit the release of most or all of the active ingredient over a short period of time, such as 60 minutes or less, and make rapid absorption of the drug possible. In contrast, extended release solid oral dosage forms permit the release of the active ingredient over an extended period of time in an effort to maintain therapeutically effective plasma levels over similarly extended time intervals, improve dosing compliance, and/or to modify other pharmacokinetic properties of the active ingredient.

“Pharmaceutically acceptable excipient” refers to a broad range of ingredients that may be combined with a compound or salt of the present invention to prepare a pharmaceutical composition or formulation. Excipients are additives that are included in a formulation because they either impart or enhance the stability, delivery and manufacturability of a drug product, and are physiologically innocuous to the recipient thereof. Regardless of the reason for their inclusion, excipients are an integral component of a drug product and therefore need to be safe and well tolerated by patients. Given the teachings and guidance provided herein, those skilled in the art will readily be able to vary the amount or range of excipient without increasing viscosity to an undesirable level. Excipients may be chosen to achieve a desired bioavailability, desired stability, resistance to aggregation or degradation or precipitation, protection under conditions of freezing, lyophilization or high temperatures, or other properties. Typically, excipients include, but are not limited to, diluents, colorants, vehicles, anti-adherants, glidants, disintegrants, flavoring agents, coatings, binders, sweeteners, lubricants, sorbents, preservatives, and the like. Examples of suitable excipients are well known to the person skilled in the art of tablet formulation and may be found e.g. in Handbook of Pharmaceutical Excipients (eds. Rowe, Sheskey & Quinn), 6th edition 2009.

As used herein the term “excipients” is intended to refer to inter alia basifying agents, solubilizers, glidants, fillers, binders, lubricant, diluents, preservatives, surface active agents, dispersing agents and the like. The term also includes agents such as sweetening agents, flavoring agents, coloring agents and preserving agents. Such components will generally be present in admixture within the tablet.

Examples of solubilizers include, but are not limited to, ionic surfactants (including both ionic and non-ionic surfactants) such as sodium lauryl sulfate, cetyltrimethylammonium bromide, polysorbates (such as polysorbate 20 or 80), poloxamers (such as poloxamer 188 or 207), and macrogols.

Examples of lubricants, glidants and flow aids include, but are not limited to, magnesium stearate, calcium stearate, stearic acid, hydrogenated vegetable oil, glyceryl palmitostearate, glyceryl behenate, sodium stearyl fumarate, colloidal silicon dioxide, and talc. The amount of lubricant in a tablet can generally be between 0.1-5% by weight.

Examples of disintegrants include, but are not limited to, starches, celluloses, cross-linked PVP, sodium starch glycolate, croscarmellose sodium, etc.

Examples of fillers (also known as bulking agents or diluents) include, but are not limited to, starches, maltodextrins, polyols (such as lactose), and celluloses. Tablets provided herein may include lactose and/or microcrystalline cellulose. Lactose can be used in anhydrous or hydrated form (e.g. monohydrate), and is typically prepared by spray drying, fluid bed granulation, or roller drying.

Examples of binders include, but are not limited to, cross-linked PVP, HPMC, microcrystalline cellulose, sucrose, starches, etc.

In some embodiments, the pharmaceutically acceptable excipients can comprise one or more diluent, binder, or disintegrant. In embodiments, the pharmaceutically acceptable excipients can comprise a diluent comprising one or more of microcrystalline cellulose, starch, dicalcium phosphate, lactose, sorbitol, mannitol, sucrose, and methyl dextrins, a binder comprising one or more of povidone, hydroxypropyl methylcellulose, hydroxypropyl cellulose, and sodium carboxymethylcellulose, and a disintegrant comprising one or more of crospovidine, sodium starch glycolate, and croscarmellose sodium.

Tablets provided herein may be uncoated or coated (in which case they include a coating). Although uncoated tablets may be used, it is more usual to provide a coated tablet, in which case a conventional non-enteric coating may be used. Film coatings are known in the art and can be composed of hydrophilic polymer materials, but are not limited to, polysaccharide materials, such as hydroxypropylmethyl cellulose (HPMC), methylcellulose, hydroxyethyl cellulose (HEC), hydroxypropyl cellulose (HPC), poly(vinylalcohol-co-ethylene glycol) and other water soluble polymers. Though the water soluble material included in the film coating of the present invention may include a single polymer material, it may also be formed using a mixture of more than one polymer. The coating may be white or colored e.g. gray. Suitable coatings include, but are not limited to, polymeric film coatings such as those comprising polyvinyl alcohol e.g. ‘Opadry® II’ (which includes part-hydrolysed PVA, titanium dioxide, macrogol 3350 and talc, with optional coloring such as iron oxide or indigo carmine or iron oxide yellow or FD&C yellow #6). The amount of coating will generally be between 2-4% of the core's weight, and in certain specific embodiments, 3%. Unless specifically stated otherwise, where the dosage form is coated, it is to be understood that a reference to % weight of the tablet means that of the total tablet, i.e. including the coating.

The pharmaceutical formulations disclosed herein can further comprise a surfactant. As used herein, the surfactant can be cationic, anionic, or non-ionic. In some embodiments, the pharmaceutical formulation can comprise a non-ionic surfactant. In some embodiments, the surfactant can comprise a polysorbate, a poloxamer, or a combination thereof. In some embodiments, the surfactant can comprise polysorbate 20, polysorbate 60, polysorbate 80, or a combination thereof.

### Methods of Treating a Subject

Further provided herein are methods of treating a subject suffering from cancer, comprising administering to the subject a therapeutically effective amount of an amorphous or crystalline form as disclosed herein, optionally as a pharmaceutical formulation as disclosed herein. In some embodiments, the cancer is multiple myeloma, non-Hodgkin's lymphoma, or acute myeloid leukemia.

### Preparation of Crystalline Forms

The crystalline forms disclosed herein can be prepared by a variety of methods known to those of skill in the art. For example, the crystalline forms can be prepared from amorphous, crude, or another crystalline form of AMG 176. In some embodiments, AMG 176 is combined with a solvent to form a desired crystalline form, for example as discussed in the examples below. In some embodiments, AMG 176 is dissolved in a solvent, or is combined with a solvent to form a slurry. In some embodiments, AMG 176 is combined with a solvent and the solution or slurry thus formed is aged to form the crystalline forms. In some embodiments, the solution or slurry is heated prior to aging or crystal formation.

### Other Embodiments

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims.

### EXAMPLES

The following examples are provided for illustration and are not intended to limit the scope of the invention.

### Materials and Methods

Commercially available reagents are used as is without further purification unless specified.

The synthesis of the starting material (AMG 176) for the following methods is disclosed in U.S. Pat. No. 9,562,061. The crystalline forms disclosed herein may be characterized using conventional means, including physical constants and spectral data.

X-Ray Powder Diffraction: X-ray powder diffraction data were obtained using a PANalytical X-Pert Pro diffractometer. The radiation used was CuKα (1.542 ⊂) with voltage and current of 45 kV and 40 mA. Data was collected at ambient temperature from 5.00 to 40.00° 2 θ using a step size of 0.0167°. A low background sample holder was used and the stage was rotated at a revolution time of 2.0 seconds. The incident beam path was equipped with a 0.02 rad soller slit, 15 mm mask, 4° fixed antiscatter slit and a programmable divergence slit. The diffracted beam was equipped with a 0.02 rad soller slit, programmable anti-scatter slit and a 0.02 mm nickel filter.

Alternatively, XRPD patterns were collected in transmission mode with a PANalytical X′Pert PRO MPD diffractometer using an incident beam of Cu radiation produced using an Optix long, fine-focus source. An elliptically graded multilayer mirror was used to focus Cu Kα X-rays through the specimen and onto the detector. Prior to the analysis, a silicon specimen (NIST SRM 640e) was analyzed to verify the observed position of the Si 111 peak is consistent with the NIST-certified position. A specimen of the sample was sandwiched between 3-μm-thick films and analyzed in transmission geometry. A beam-stop, short antiscatter extension and antiscatter knife edge were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v. 2.2b. The data acquisition parameters for each pattern are displayed above the image in the Data section of this report including the divergence slit (DS) before the mirror.

XRPD patterns were collected in reflection mode with a PANalytical X'Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was packed in a well. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 2.2b. The data acquisition parameters for each pattern are displayed above the image in the Data section of this report including the divergence slit (DS) and the incident-beam SS.

Within the figure referenced for a given indexed XRPD pattern, agreement between the allowed peak positions, marked with red bars, and the observed peaks indicates a consistent unit cell determination. Successful indexing of a pattern indicates that the sample is composed primarily of a single crystalline phase unless otherwise stated. Space groups consistent with the assigned extinction symbol, unit cell parameters, and derived quantities are tabulated below the figure. To confirm the tentative indexing solution, the molecular packing motifs within the crystallographic unit cells must be determined. No attempts at molecular packing were performed.

Single Crystal Structure: A colorless needle crystal (monohydrate) with dimensions 0.18×0.11×0.05 mm was mounted on a Nylon loop using very small amount of paratone oil. The crystal was mounted, cell determined to be the monohydrate, and then the temperature was raised to 400 ° K to dehydrate. The crystal remained at 400 ° K for 4 hours, and then lowered temperature to 173 ° K and data collected. Data were collected using a Bruker CCD (charge coupled device) based diffractometer equipped with an Oxford Cryostream low-temperature apparatus operating at 173° K. Data were measured using omega and phi scans of 0.5° per frame for 30 s. The total number of images was based on results from the program COSMO where redundancy was expected to be 4.0 and completeness to 100% out to 0.83 Å. Cell parameters were retrieved using APEX II software and refined using SAINT on all observed reflections. Data reduction was performed using the SAINT software. Scaling and absorption corrections were applied using SADABS multi-scan technique. The structures are solved by the direct method using the SHELXS-97 program and refined by least squares method on F2, SHELXL-97, which are incorporated in SHELXTL-PC V 6.10.

Differential Scanning calorimetry: Differential scanning calorimetry (DSC) analysis was conducted on a TA Instruments Q100 instrument. A sample size of approximately 1 mg was weighed out into a standard aluminum DSC pan; the pan was uncrimped. The sample was heated at 10° C./min from ambient temperature to 300° C. under dry nitrogen at 50 mL/min. Modulated DSC analysis was conducted using a TA Instruments Q100 instrument. Sample sizes of approximately 1 mg were used in aluminum, uncrimped pan. The samples were equilibrated at 20° C. and held for 5 minutes before heating to 300° C. at a heating rate of 3° C./min under dry nitrogen at 50 mL/min. Modulation was ±0.75° C. every 45 seconds.

Alternatively, DSC was performed using a TA Instruments Q2000 differential scanning calorimeter.

Temperature calibration was performed using NIST-traceable indium metal. The sample was placed into an aluminum Tzero pan, covered with a lid, crimped, and the weight was accurately recorded. A weighed aluminum pan configured as the sample pan was placed on the reference side of the cell. The data acquisition parameters and pan configuration for each thermogram are displayed in the image in the Data section of this report. The method code on the thermogram is an abbreviation for the start and end temperature as well as the heating rate; e.g., −30-250-10 means “from −30° C. to 250° C., at 10° C./min”.

Alternatively, MDSC data were obtained on a TA Instruments Q2000 differential scanning calorimeter equipped with a refrigerated cooling system (RCS). Temperature calibration was performed using NIST-traceable indium metal. The sample was placed into an aluminum DSC pan, and the weight was accurately recorded. The pan was covered with a lid and the lid was crimped. A weighed, crimped aluminum pan was placed on the reference side of the cell. Data were obtained using a modulation amplitude of ±0.8° C. and a 60 second period with an underlying heating rate of 2° C./minute from ±30° C. to 250° C. The reported glass transition temperature is obtained from the inflection point of the step change in the reversing heat flow versus temperature curve.

Alternatively, DSC was performed using a Mettler-Toledo DSC3+ differential scanning calorimeter. Temperature calibration was performed using adamantane, phenyl salicylate, indium, tin, and zinc. The sample was placed into a hermetically sealed or an open aluminum DSC pan, and the weight was accurately recorded. A weighed aluminum pan configured as the sample pan was placed on the reference side of the cell. The samples were analyzed from −30 to 250° C. at a ramp rate of 10° C./min. Although thermograms are plotted by reference temperature (x-axis), results are reported according to sample temperatures.

Thermal Analysis: Thermogravimetric analysis was conducted on a TA Instruments Q500 instrument. A sample size of approximately 1-5 mg was used in an aluminum pan. The sample was heated at 10° C./min from ambient temperature to 400° C. under dry nitrogen at 25 mL/min.

Alternatively, TG analyses were performed using a TA Instruments Q5000 IR thermogravimetric analyzer. Temperature calibration was performed using nickel and Alumel™. Each sample was placed in an aluminum pan. The sample was hermetically sealed, the lid pierced, then inserted into the TG furnace. The furnace was heated under nitrogen. The data acquisition parameters for each thermogram are displayed in the image in the Data section of this report. The method code on the thermogram is an abbreviation for the start and end temperature as well as the heating rate; e.g., 25-350-10 means “from 25° C. to 350° C., at 10° C./min”.

Alternatively, Thermogravimetric analyses were performed using a Mettler Toledo TGA/DSC3+ analyzer. Temperature calibration was performed using phenyl salicylate, indium, tin, and zinc. The sample was placed in an aluminum pan. The open pan was inserted into the TG furnace. The furnace was heated under nitrogen. Each sample was heated from ambient temperature to 350° C. at ramp rates of 2, 5, or 10° C./min. Although thermograms are plotted by reference temperature (x-axis), results are reported according to sample temperatures.

Moisture Sorption: Moisture sorption data was collected at 25° C. using a VTI vapor sorption analyzer. A sample size of approximately 4-10 mg was used in a standard platinum pan. Hygroscopicity was evaluated from 5 to 95% RH in increments of 5% RH. Data for adsorption and desorption cycles were collected. Equilibrium criteria were set at 0.001% weight change in 10 minute with a maximum equilibration time of 180 minutes.

NMR: Solution proton NMR spectra were acquired by Spectral Data Services of Champaign, IL at 25° C. with a Varian UNITYINOVA-400 spectrometer. Samples were dissolved in DMSO-d6. In some cases, the solution NMR spectra were acquired at SSCI with an Agilent DD2-400 spectrometer using deuterated DMSO or methanol.

¹³C SSNMR data was collected on a Bruker DSX spectrometer operating at 600 MHz (¹H). A 4 mm H/F/X spinning probe operating at a spinning frequency of 14 kHz was used for all experiments. CPMAS with TOSS program was used with a recycle delay of 10 s. A ¹H 90° pulse of 2.5 ps and ¹³C 180° pulse of 8 μs were used. Decoupling was carried out using a spina164 sequence. 4096 transients were acquired for signal averaging. The data was processed with Topspin 3.0 software.

### Example 1: AMG 176 Form 1

AMG 176 Form 1 has acceptable pharmaceutical and physical properties, can be scaled to kilogram quantities, and can be compounded during drug product formulating as an in situ sodium salt to achieve desired drug concentrations to enable IV delivery. AMG 176 can also be converted to amorphous material to support oral dosing, if desired, depending on the dosing range. Dog PK studies suggested that absorption was improved by 10× when AMG 176 was dosed as an amorphous suspension as compared to the crystalline suspension. While improved exposures were observed by dosing the amorphous material, the exposures would likely plateau if gram level doses are need for oral delivery.

AMG 176 (121 g) was dissolved in ethyl acetate. Ethanol (800 mL) was added, and the combination was mixed for 20 min while heating. Water (250 mL) was added dropwise over 30 min. The slurry was allowed to cool to room temperature for 2 h and then further cooled in an ice bath for 4 h prior to filtering. The wet cake was washed with cold 30% ethanol in water (300 mL). The cake was air dried for two days and then further dried under vaccum at 40° C. for four days. Dried solids were identified as crystalline AMG 176 Form 1.

AMG 176 Form 1 consists of anhydrous/non-solvated AMG 176 and is likely thermodynamically stable between 2 and 79° C. An XRPD pattern for Form 1 was successfully indexed, indicating the material consists primarily or exclusively of a single crystalline phase. The unit cell volume obtained from the indexing solution contains minimal free volume, consistent with anhydrous/non-solvated AMG 176. A proton NMR spectrum for Form 1 was measured in deuterated DMSO. The spectrum was consistent with the chemical structure of AMG 176.

AMG 176 Form 1 was analyzed by DSC and TGA. Negligible weight loss is observed by TGA up to 220° C., consistent with an anhydrous/non-solvated material (FIG. 4). The DSC thermogram is uneventful until the onset of a sharp endotherm at 232° C., likely corresponding with simultaneous melting and decomposition (FIG. 3).

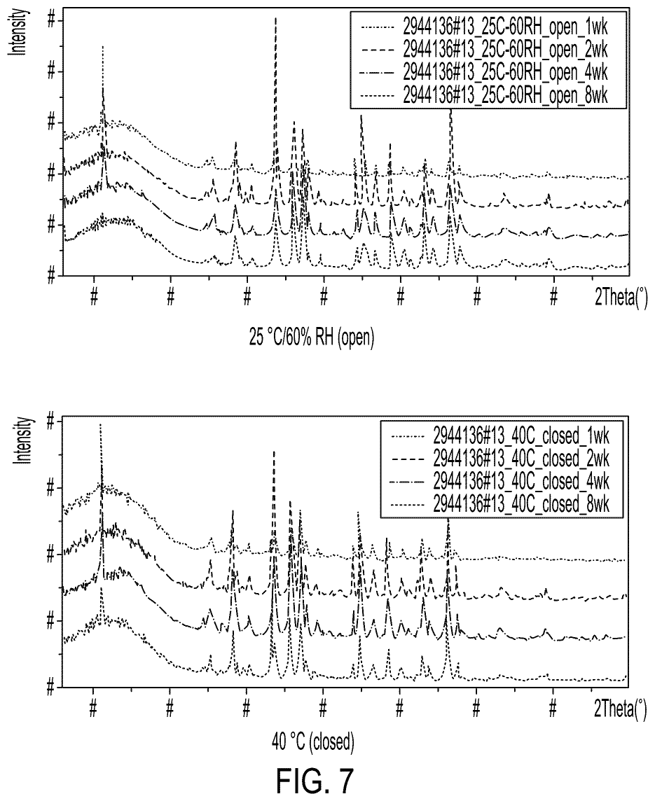

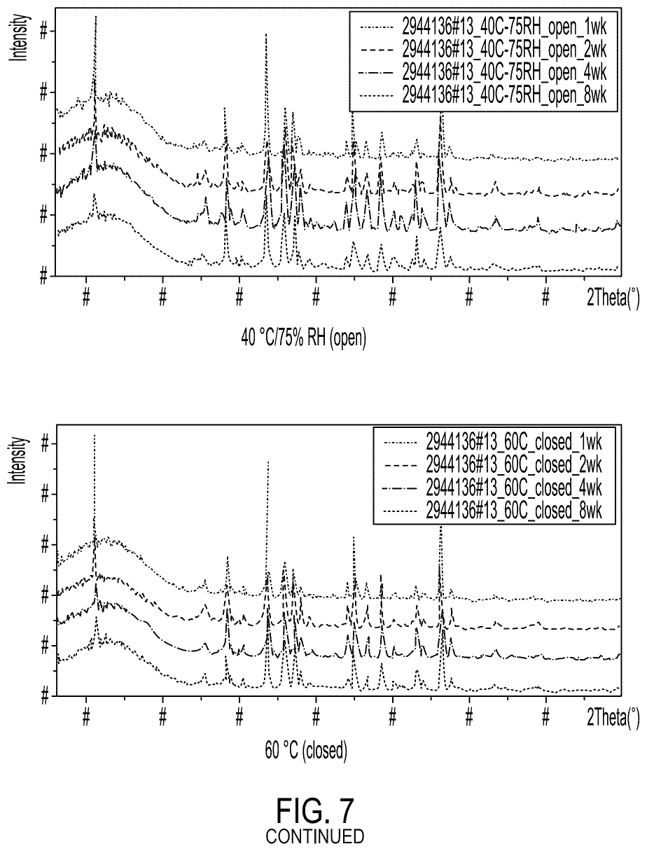

AMG 176 Form 1 was also analyzed by DVS (FIG. 5). The material exhibited low hygroscopicity, taking up only 0.3% water vapor between 5% and 95% RH. All of this weight was lost upon desorption with virtually no hysteresis observed. XRPD of the post-DVS material indicated no form change (FIG. 7).

TABLE 1
XRPD Data Table

| | | | | Rel. |
|---|---|---|---|---|
| Pos. [°2Th.] | FWHM [°2Th.] | d-spacing [Å] | Height [cts] | Int. [%] |
| 5.62 | 0.16 | 15.72 | 1159.12 | 16.00 |
| 8.35 | 0.19 | 10.58 | 155.73 | 2.15 |
| 12.30 | 0.16 | 7.20 | 1395.51 | 19.27 |
| 12.61 | 0.10 | 7.02 | 1932.24 | 26.68 |
| 12.81 | 0.10 | 6.91 | 1368.50 | 18.89 |
| 13.54 | 0.16 | 6.54 | 537.81 | 7.43 |
| 14.19 | 0.16 | 6.24 | 4709.46 | 65.02 |
| 14.50 | 0.10 | 6.11 | 1322.25 | 18.26 |
| 14.94 | 0.16 | 5.93 | 711.49 | 9.82 |
| 15.28 | 0.19 | 5.80 | 1042.99 | 14.40 |
| 16.69 | 0.19 | 5.31 | 1111.37 | 15.34 |
| 17.04 | 0.16 | 5.20 | 1883.03 | 26.00 |
| 17.98 | 0.26 | 4.93 | 7243.22 | 100.00 |
| 18.64 | 0.19 | 4.76 | 6735.32 | 92.99 |
| 19.05 | 0.13 | 4.66 | 1038.67 | 14.34 |
| 19.69 | 0.16 | 4.51 | 1028.18 | 14.20 |
| 21.05 | 0.29 | 4.22 | 197.66 | 2.73 |
| 22.11 | 0.16 | 4.02 | 960.66 | 13.26 |
| 22.72 | 0.16 | 3.91 | 1925.83 | 26.59 |
| 23.40 | 0.16 | 3.80 | 584.67 | 8.07 |
| 24.36 | 0.19 | 3.65 | 1468.17 | 20.27 |
| 25.21 | 0.23 | 3.53 | 1734.61 | 23.95 |
| 25.74 | 0.16 | 3.46 | 454.24 | 6.27 |
| 26.31 | 0.10 | 3.39 | 645.73 | 8.91 |
| 26.62 | 0.19 | 3.35 | 2615.20 | 36.11 |
| 27.10 | 0.19 | 3.29 | 1186.83 | 16.39 |
| 28.10 | 0.10 | 3.18 | 850.14 | 11.74 |
| 28.29 | 0.10 | 3.15 | 1107.43 | 15.29 |
| 28.86 | 0.13 | 3.09 | 1138.69 | 15.72 |
| 29.20 | 0.13 | 3.06 | 667.56 | 9.22 |
| 30.18 | 0.23 | 2.96 | 206.09 | 2.85 |
| 30.88 | 0.16 | 2.90 | 275.11 | 3.80 |
| 31.57 | 0.10 | 2.83 | 373.63 | 5.16 |
| 32.32 | 0.39 | 2.77 | 335.44 | 4.63 |
| 32.78 | 0.13 | 2.73 | 309.44 | 4.27 |
| 33.73 | 0.19 | 2.66 | 249.09 | 3.44 |
| 34.16 | 0.13 | 2.62 | 421.08 | 5.81 |
| 36.17 | 0.45 | 2.48 | 120.94 | 1.67 |
| 38.71 | 0.19 | 2.33 | 266.03 | 3.67 |
| 39.87 | 0.19 | 2.26 | 342.99 | 4.74 |
| 40.25 | 0.19 | 2.24 | 348.15 | 4.81 |
| 41.27 | 0.29 | 2.19 | 769.25 | 10.62 |
| 42.75 | 0.32 | 2.11 | 280.57 | 3.87 |

Single Crystal Data: The crystal structure of AMG 176 Form 1 was determined. The crystal parameters are summarized in Table 2 and the unit cell is displayed in FIG. 6.

TABLE 2
X-ray Single Structure Data

| | |
|---|---|
| Crystal System | Orthorhombic |
| Space Group | P2₁2₁2₁ |
| Unit Cell | a = 6.964 Å |
| | b = 14.402 Å |
| | c = 31.391 Å |
| | α = β = γ = 90° |
| Volume | 3148 Å³ |
| Z | 4 |
| Density (calculated) | 1.294 g/cm³ |

Solid state stability assessment of AMG 176 Form 1 was conducted at 25° C./60% RH (open), 40° C. (closed), 60° C. (closed), and 40° C./75% RH (open) accelerated conditions to determine the chemical and physical stability of AMG 176. After 8 weeks, Form 1 was physically and chemically stable at all stress conditions, as displayed in FIG. 7.

### Example 2: AMG 176 Form 2

AMG 176 Form 2 consists of an isostructural solvate containing 1 mole of 2-MeTHF, THF, MTBE, or 1,4-dioxane per mole of AMG 176. The form was produced from numerous slurry or vapor stress experiments in relevant solvent systems. A representative sample from each solvent was characterized by XRPD indexing, proton NMR, DSC, and TGA for comparison. The solvent systems that produced AMG 176 Form 2 were IPA/heptane, 2-MeTHF, THF/water, MTBE, and 1,4-dioxane/water.

Peak shifting was observed among the XRPD patterns, consistent with an isostructural solvate. The unit cell volume from the XRPD indexing solutions and the proton NMR spectra were all consistent with approximately 1 mole of the respective solvent in the crystal lattice. Additionally, the quantity of weight loss in each of the TGA thermograms is consistent with the loss of between 0.8 and 1 mole of respective solvent. Desolvation events occur at slightly different onsets based on endotherms observed by DSC, and an overlay of DSC and TGA thermograms for each sample is presented in FIGS. 9-13 to illustrate differences in weight loss upon heating. The DSC thermograms for the samples from 2-MeTHF and 1,4-dioxane exhibit an exothermic event accompanying the desolvation endotherm, likely indicating form transformation.

Based on the DSC and TGA data, experiments were designed to explore the desolvation of various AMG 176 Form 2 samples by heating, vacuum, and VT-XRPD. Attempts to desolvate samples of Form 2 under vacuum at about 70° C. and upon heating at about 164-165° C. for 10 minutes were insufficient in removing the solvent.

An attempt to reproduce Form 2 by slurrying in 2-MeTHF at about 600 mg scale was successful. The material was utilized for form screen experiments.

For example, AMG 176 Form 2 was formed by slurrying AMG 176 in IPA/heptane (1:1, 8.4 mg/mL) at 25° C. for 8 h. Solvent was allowed to evaporate. Remaining solids were identified as crystalline Form 2 by XRPD.

TABLE 3
XRPD Data Table

| | | | | Rel. |
|---|---|---|---|---|
| Pos. [°2Th.] | FWHM [°2Th.] | d-spacing [Å] | Height [cts] | Int. [%] |
| 12.99 | 0.15 | 6.82 | 1415.50 | 59.05 |
| 16.90 | 0.15 | 5.25 | 2397.27 | 100.00 |
| 17.25 | 0.10 | 5.14 | 565.60 | 23.59 |
| 19.10 | 0.15 | 4.65 | 108.70 | 4.53 |
| 21.46 | 0.30 | 4.14 | 56.61 | 2.36 |
| 22.67 | 0.30 | 3.92 | 72.39 | 3.02 |
| 24.32 | 0.15 | 3.66 | 89.22 | 3.72 |
| 26.95 | 0.39 | 3.31 | 38.12 | 1.59 |
| 33.80 | 0.15 | 2.65 | 46.86 | 1.95 |
| 40.03 | 0.30 | 2.25 | 27.97 | 1.17 |
| 42.84 | 0.36 | 2.11 | 33.89 | 1.41 |

A summary of differentiating XRPD peaks between AMG Form 1 and Form 2 is presented in Table 4 below.

TABLE 4

| Free Base Form | Peaks Unique to Each Form (KA1°) | | | | | |
|---|---|---|---|---|---|---|
| Form 1 | 12.30 | 12.61 | 13.54 | 14.19 | 14.94 | 15.28 |
| Form 2 | 12.99 | 17.25 | 21.46 | | | |

Example 3: Amorphous AMG 176

Amorphous material resulted from numerous evaporation and vapor diffusion experiments during a form screen. Noting the ease of preparation by fast evaporation, the material was prepared on a about 500 mg scale from DCM for use in additional form screen experiments and characterization.

The amorphous material was characterized by proton NMR, DVS, modulated DSC, and TGA. The proton NMR spectrum was consistent with the chemical structure of AMG 176.

The DVS isotherm is shown in FIG. 18. The amorphous material exhibited limited hygroscopicity, taking up 0.95 wt % water vapor between 5% and 95% RH. More weight was lost upon desorption (1.59%) than was gained on adsorption, likely due to the loss of residual DCM and/or water from the starting material. XRPD of the post-DVS material was consistent with amorphous material.

The observation of a glass transition (Tg) can be characteristic of the non-crystalline nature of the material. Modulated DSC (mDSC) was performed to determine the Tg of the material and is shown in FIG. 15. mDSC permits separation of the total heat flow signal into its thermodynamic (heat capacity) and kinetic components. Therefore, the Tg can typically be seen as a step change in the reversing signal. Amorphous AMG 176 exhibits a Tg at approximately 163° C. (ACP: 0.3 J/(g ° C.)). To be noted, differences in solvent and/or water content within the sample can shift the temperature at which the glass transition occurs.

A TGA thermogram is shown in FIG. 17. Gradual weight loss of 3.8 wt % was observed between 28 and 180° C., consistent with the loss solvent (likely DCM and water).

Attempts to crystallize amorphous material were set up by vapor stressing and slurrying under various conditions. Most of the experiments caused crystallization to Form 1, with the exception of those that resulted in Form 2 from relevant solvent systems (1,4-dioxane/water, 2-MeTHF, and MTBE). Only one experiment, vapor stress with water at RT, did not cause crystallization, likely due to a lack of sufficient solubility.

For example, Amorphous AMG 176 was formed by dissolving AMG 176 (3.9 g) in a minimal amount of EtOAc then rapidly precipitating with heptane. The suspension was filtered. Remaining solids were identified as amorphous by XRPD.

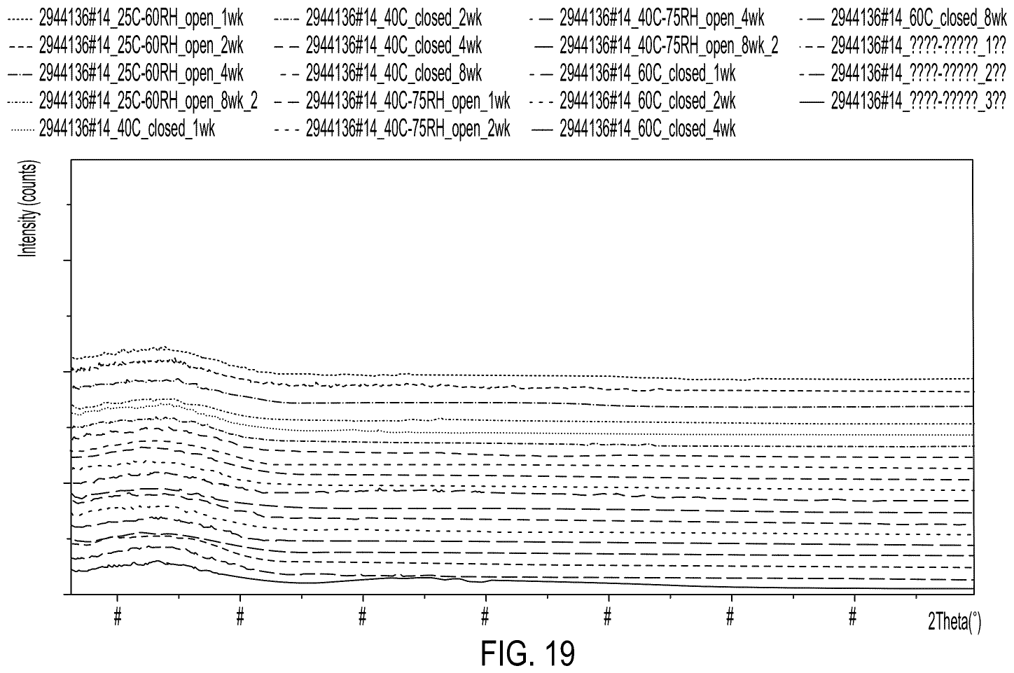

Solid state stability assessment of AMG 176 amorphous material and Form 1 was conducted at 25° C./60% RH (open), 40° C. (closed), 60° C. (closed), and 40° C./75% RH (open) accelerated conditions to determine the chemical and physical stability of AMG 176. After 8 weeks, the amorphous material and Form 1 were physically and chemically stable at all stress conditions, as displayed in FIG. 19.

### Example 4: AMG 176 Calcium Hydrate Form 1

AMG 176 sodium salt was dissolved in ethanol at 140 mg/mL to which 0.55eq CaCl².2H₂O was added as a solution in ethanol resulting in a precipitate. The suspension was filtered and then a 0.5 mL aliquot of the filtrate was layered with heptane and then left at room temperature to crystallize. Solids were identified as crystalline by XRPD (FIG. 20).

TGA analysis showed approximately 5.5% weight loss that was confirmed to be due to the loss of water and ethanol by TGA-IR. Approximately 0.5 molar equivalent of Ca was detected. The Ca salt appeared to melt at approximately 301° C., recrystallize, and undergo another melt at 322° C. followed by degradation. The TGA and DSC data are presented in FIG. 21.

TABLE 5
XRPD Data Table

| | | | | Rel. |
|---|---|---|---|---|
| Pos. [°2Th.] | FWHM [°2Th.] | d-spacing [Å] | Height [cts] | Int. [%] |
| 6.77 | 0.16 | 13.06 | 7244.50 | 100 |
| 7.76 | 0.10 | 11.39 | 2682.06 | 37.02 |
| 11.64 | 0.13 | 7.60 | 822.86 | 11.36 |
| 15.54 | 0.10 | 5.70 | 2559.39 | 35.33 |
| 19.44 | 0.19 | 4.57 | 213.72 | 2.95 |
| 31.79 | 0.19 | 2.82 | 119.34 | 1.65 |

AMG 176 calcium hydrate Form 1—¹H NMR confirmed salt formation with residual ethanol. TGA analysis showed approximately 5.5% weight loss from 25-225° C. that was confirmed to be due to the loss of water and ethanol by TGA-IR. Approximately 0.5 molar equivalent of Ca was detected by CE.

1H NMR (400 MHz, DMSO-d⁶) δ 6 ppm 0.78-1.04 (m, 4H) 1.04-1.18 (m, 4H) 1.18-1.29 (m, 1H) 1.29-1.53 (m, 2H) 1.54-1.79 (m, 4H) 1.84 (br d, J=7.67 Hz, 3H) 1.91-2.11 (m, 4H) 2.22-2.49 (m, 3H) 2.65-2.83 (m, 2H) 2.89-3.07 (m, 1H) 3.07-3.12 (m, 3H) 3.17 (br d, J=14.10 Hz, 1H) 3.34-3.60 (m, 6H) 3.64-3.82 (m, 2H) 3.82-4.06 (m, 5H) 4.35 (t, J=5.08 Hz, 1H) 5.36 (br dd, J=15.45, 9.23 Hz, 2H) 6.00 (br s, 2H) 6.73 (d, J=8.09 Hz, 2H) 6.88-6.96 (m, 2H) 7.01 (dd, J=7.98, 1.76 Hz, 2H) 7.16 (d, J=2.28 Hz, 2H) 7.27 (dd, J=8.50, 2.28 Hz, 2H) 7.69 (d, J=8.71 Hz, 1H).

### Example 5: AMG 176 Calcium Hydrate Form 2

AMG 176 sodium was dissolved in EtOH at 43.6 mg/mL to which 0.55eq CaCl₂.2H₂O was added as a solution in ethanol. To the filtrate seeds of AMG 176 calcium Form 1 were added then stirred at room temperature. Solids were identified as crystalline by XRPD (FIG. 22).

AMG 176 Ca salt Form 2 has a weight loss of approximately 3.6% by TGA (FIG. 30), consistent with 3.2% presence of water. Moisture sorption analysis showed a weight gain of approximately 5.2% at 70% RH and 23% weight gain at 95% RH, which is likely due to the presence of residual NaCl (FIG. 23). The Ca content was determined to be 3.17%, which corresponds to approximately 0.5 mole of Ca. Aqueous solubility of this crystalline salt was determined to be approximately 24 μg/mL.

TABLE 6
XRPD Data Table

| | | | | Rel. |
|---|---|---|---|---|
| Pos. [°2Th.] | FWHM [°2Th.] | d-spacing [Å] | Height [cts] | Int. [%] |
| 6.18 | 0.16 | 14.31 | 6355.17 | 100.00 |
| 7.85 | 0.16 | 11.27 | 1083.73 | 17.05 |
| 8.30 | 0.16 | 10.65 | 660.95 | 10.40 |
| 9.73 | 0.19 | 9.09 | 397.92 | 6.26 |
| 10.11 | 0.19 | 8.75 | 726.66 | 11.43 |
| 10.83 | 0.26 | 8.17 | 2221.33 | 34.95 |
| 11.79 | 0.26 | 7.50 | 2055.65 | 32.35 |
| 13.08 | 0.23 | 6.77 | 515.29 | 8.11 |
| 13.93 | 0.26 | 6.36 | 2081.42 | 32.75 |
| 14.75 | 0.19 | 6.01 | 628.86 | 9.90 |
| 15.49 | 0.26 | 5.72 | 2516.87 | 39.60 |
| 16.42 | 0.19 | 5.40 | 1013.66 | 15.95 |
| 16.88 | 0.19 | 5.25 | 1923.11 | 30.26 |
| 17.51 | 0.23 | 5.06 | 2121.53 | 33.38 |
| 18.23 | 0.23 | 4.87 | 2842.52 | 44.73 |
| 19.29 | 0.32 | 4.60 | 3208.45 | 50.49 |
| 19.61 | 0.10 | 4.53 | 1955.58 | 30.77 |
| 20.19 | 0.26 | 4.40 | 4717.35 | 74.23 |
| 20.88 | 0.23 | 4.25 | 665.88 | 10.48 |
| 21.58 | 0.29 | 4.12 | 2527.73 | 39.77 |
| 22.71 | 0.26 | 3.92 | 1474.97 | 23.21 |
| 23.27 | 0.13 | 3.82 | 1745.80 | 27.47 |
| 23.47 | 0.13 | 3.79 | 1704.15 | 26.82 |
| 24.40 | 0.29 | 3.65 | 3880.12 | 61.05 |
| 25.53 | 0.23 | 3.49 | 2165.24 | 34.07 |
| 26.02 | 0.19 | 3.42 | 2218.48 | 34.91 |
| 26.46 | 0.32 | 3.37 | 1335.35 | 21.01 |
| 27.40 | 0.16 | 3.25 | 957.94 | 15.07 |
| 28.01 | 0.19 | 3.19 | 1047.73 | 16.49 |
| 28.90 | 0.26 | 3.09 | 871.40 | 13.71 |
| 29.96 | 0.29 | 2.98 | 1157.74 | 18.22 |
| 30.40 | 0.19 | 2.94 | 615.78 | 9.69 |
| 31.91 | 0.29 | 2.80 | 1232.90 | 19.40 |
| 33.14 | 0.26 | 2.70 | 478.62 | 7.53 |
| 33.81 | 0.26 | 2.65 | 408.33 | 6.43 |
| 34.60 | 0.23 | 2.59 | 153.05 | 2.41 |
| 35.30 | 0.26 | 2.54 | 308.42 | 4.85 |
| 35.93 | 0.39 | 2.50 | 275.99 | 4.34 |
| 39.46 | 0.45 | 2.28 | 262.55 | 4.13 |
| 40.08 | 0.19 | 2.25 | 70.29 | 1.11 |
| 40.92 | 0.32 | 2.21 | 479.29 | 7.54 |
| 42.36 | 0.26 | 2.13 | 293.35 | 4.62 |
| 42.97 | 0.32 | 2.10 | 419.54 | 6.60 |

AMG 176 calcium hydrate Form 2—¹H NMR confirmed salt formation with residual ethanol. A 3.6% weight loss was seen on TGA from 25-225° C. This was confirmed to be water by KF (3.2%). The Ca content was determined to be 3.17% by capillary electrophoresis which corresponds to 0.5 mol calcium.

1H NMR (400 MHz, DMSO-d₆) δ ppm 0.85 (d, J=6.01 Hz, 3H) 0.99-1.21 (m, 5H) 1.24-1.44 (m, 1H) 1.55-1.79 (m, 3H) 1.84 (br d, J=7.46 Hz, 3H) 1.91-2.09 (m, 4H) 2.22-2.48 (m, 2H) 2.65-2.83 (m, 2H) 2.89-3.07 (m, 1H) 3.09 (s, 2H) 3.13-3.31 (m, 1H) 3.34-3.60 (m, 4H) 3.72 (br d, J=14.93 Hz, 1H) 3.85-4.03 (m, 3H) 4.35 (t, J=5.08 Hz, 1H) 5.36 (dd, J=15.34, 9.12 Hz, 1H) 6.01 (br dd, J=15.65, 8.40 Hz, 1H) 6.72 (d, J=8.09 Hz, 1H) 6.91-6.97 (m, 1H) 7.01 (dd, J=7.98, 1.76 Hz, 2H) 7.16 (d, J=2.28 Hz, 2H) 7.27 (dd, J=8.50, 2.28 Hz, 2H) 7.70 (d, J=8.71 Hz, 1H).

### Example 6: AMG 176 Calcium Hydrate Form 3

AMG 176 calcium salt Form 2 was slurried at 17.5 mg/mL in water for 24 h at 25° C. Solids were identified as crystalline by XRPD.

Approximately 3.5% weight is lost upon TGA analysis (FIG. 26), which is likely due to the loss of water based on KF results, 4.3% water. A melting point of approximately 314° C. was observed by DSC analysis (FIG. 25). The material was determined to be hygroscopic absorbing approximately 7.4% of moisture at 95% RH (FIG. 27). The amount of Ca present in the sample was approximately 1.8%, which was lower than expected.

TABLE 7
XRPD Data Table

| | | | | Rel. |
|---|---|---|---|---|
| Pos. [°2Th.] | FWHM [°2Th.] | d-spacing [Å] | Height [cts] | Int. [%] |
| 6.21 | 0.13 | 14.23 | 774.30 | 89.01 |
| 6.70 | 0.19 | 13.19 | 869.87 | 100.00 |
| 8.22 | 0.13 | 10.76 | 689.30 | 79.24 |
| 11.13 | 0.16 | 7.95 | 256.33 | 29.47 |
| 12.00 | 0.26 | 7.38 | 185.10 | 21.28 |
| 12.98 | 0.19 | 6.82 | 127.26 | 14.63 |
| 17.38 | 0.19 | 5.10 | 307.15 | 35.31 |
| 18.58 | 0.13 | 4.78 | 635.95 | 73.11 |
| 20.01 | 0.19 | 4.44 | 347.79 | 39.98 |
| 21.39 | 0.26 | 4.15 | 228.08 | 26.22 |
| 25.36 | 0.19 | 3.51 | 186.26 | 21.41 |
| 29.43 | 0.19 | 3.03 | 183.70 | 21.12 |

AMG 176 calcium hydrate Form 3—¹H NMR spectrum confirms salt formation and no residual organic solvent. Water content was 4.3% by KF.

¹H NMR (400 MHz, DMSO-d₆) δ ppm 0.85 (br d, J=6.01 Hz, 3H) 1.13 (d, J=7.05 Hz, 3H) 1.36 (br t, J=9.85 Hz, 1H) 1.55-1.79 (m, 3H) 1.84 (br d, J=7.46 Hz, 2H) 1.92-2.03 (m, 3H) 2.21-2.48 (m, 2H) 2.65-2.83 (m, 2H) 2.89-3.07 (m, 1H) 3.09 (s, 2H) 3.13-3.31 (m, 1H) 3.44-3.62 (m, 2H) 3.72 (br d, J=14.72 Hz, 2H) 3.86-4.06 (m, 3H) 5.36 (br dd, J=15.24, 9.02 Hz, 2H) 6.01 (br dd, J=15.13, 8.09 Hz, 1H) 6.72 (d, J=8.09 Hz, 1H) 6.92-6.97 (m, 1H) 7.01 (dd, J=8.09, 1.66 Hz, 1H) 7.16 (d, J=2.49 Hz, 1H) 7.27 (dd, J=8.50, 2.49 Hz, 1H) 7.70 (d, J=8.50 Hz, 1H).

### Example 7: Amorphous AMG 176 Calcium Hydrate

Amorphous AMG 176 Calcium Hydrate appeared as irregular shaped particles by microscopy with some minor presence of birefringence. When analyzed by XRPD, the amorphous material did not exhibit any characteristic diffraction peaks (FIG. 28). Approximately 2% weight loss was observed by TGA (FIG. 30), which is likely due to residual solvent. DSC analysis showed a melting endotherm at approximately 292° C. (FIG. 29). The amorphous material likely recrystallized during the analysis even though a clear recrystallization exotherm is not observed. Upon moisture sorption analysis, the amorphous material was determined to be hygroscopic gaining approximately 9% weight at 95% RH (FIG. 31).

The amorphous material exhibited limited hygroscopicity, taking up 0.95 wt % water vapor between 5% and 95% RH. More weight was lost upon desorption (1.59%) than was gained on adsorption, likely due to the loss of residual DCM and/or water from the starting material. XRPD of the post-DVS material was consistent with amorphous material.

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

The use of the terms “a,” “an,” “the,” and similar referents in the context of the disclosure herein (especially in the context of the claims) are to be construed to cover both the singular and the plural, unless otherwise indicated. Recitation of ranges of values herein merely are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure herein and is not a limitation on the scope of the disclosure herein unless otherwise indicated. No language in the specification should be construed as indicating any non-claimed element as essential to the practice of the disclosure herein.

All patents, publications and references cited herein are hereby fully incorporated by reference. In case of conflict between the present disclosure and incorporated patents, publications and references, the present disclosure should control.

1. A crystalline form of AMG 176,
(a) characterized by solid state ¹³C NMR peaks at 12.39, 19.40, 20.46, 27.24, 28.07, 30.54, 33.09, 33.80, 37.18, 41.80, 42.98, 54.86, 58.69, 60.01, 63.11, 80.06, 85.77, 117.20, 119.77, 120.86, 127.04, 129.01, 129.71, 131.30, 132.24, 133.58, 139.16, 140.11, 140.69, 152.08, and 169.82±0.5 ppm (“AMG 176 Form 1”) or
(b) characterized by XRPD pattern peaks at 13.0, 16.9, and 17.3±0.2° 2θ using Cu Kα radiation (“AMG 176 Form 2”).

2. The crystalline form of claim 1, wherein Form 1 is further characterized by XRPD pattern peaks at 14.2, 18.0, and 18.6±0.2° 2θ using Cu Kα radiation.

3. The crystalline form of claim 2, wherein Form 1 is further characterized by XRPD pattern peaks at 12.6, 17.0, 22.7, and 26.6±0.2° 20 using Cu Kα radiation.

4. The crystalline form of claim 3, wherein Form 1 is further characterized by XRPD pattern peaks at 5.6, 12.3, 12.8, 14.5, 16.7, 24.4, 25.2, 27.1, 28.3, and 28.9±0.2° 2θ using Cu Kα radiation.

5. (canceled)

6. The crystalline form of claim 1, having wherein Form 1 has an endothermic transition at 233° C. to 238° C., as measured by differential scanning calorimetry.

7. The crystalline form of claim 6, wherein the endothermic transition is at 236° C.±3° C.

8.-9. (canceled)

10. The crystalline form of claim 1, wherein Form 2 is further characterized by XRPD pattern peaks at 19.1, 21.5, 22.7, 24.3, 27.0, 33.8, 40.0, and 42.8±0.2° 2θ using Cu Kα radiation.

11. (canceled)

12. The crystalline form of claim 1, wherein Form 2 has an endothermic transition at 232° C. to 238° C., optionally at 162° C. to 169° C., 169° C. to 173° C., 177° C. to 179° C., or 178° C. to 182° C., as measured by differential scanning calorimetry.

13-16. (canceled)

17. An amorphous form of AMG 176, having an XRPD pattern substantially as shown in FIG. 14 (“AMG 176 Amorphous”), optionally having an endothermic transition at 122° C. to 130° C. or 159° C. to 166° C. as measured by differential scanning calorimetry;
or an amorphous form of AMG 176, as a calcium salt hydrate, having an XRPD pattern substantially as shown in FIG. 28 (“AMG 176 Amorphous Calcium Hydrate”).

18-20. (canceled)

21. A crystalline form of AMG 176, as a calcium salt hydrate,
(a) characterized by XRPD pattern peaks at 6.8, 7.8, and 15.5±0.2° 2θ using Cu Kα radiation (“AMG 176 Calcium Hydrate Form 1”) or
(b) characterized by XRPD pattern peaks at 6.2, 20.2, and 24.4±0.2° 2θ using Cu Kα radiation (“AMG 176 Calcium Hydrate Form 2”); or
(c) characterized by XRPD pattern peaks at 6.2, 6.7, and 8.2±0.2° 2θ using Cu Kα radiation (“AMG 176 Calcium Hydrate Form 3”).

22. The crystalline form of claim 21, wherein AMG 176 Calcium Hydrate Form 1 is further characterized by XRPD pattern peaks at 11.6, 19.4, and 31.8±0.2° 2θ using Cu Kα radiation.

23.-26. (canceled)

27. The crystalline form of claim 21, wherein AMG 176 Calcium Hydrate Form 2 is further characterized by XRPD pattern peaks at 15.5, 18.2, 19.3, and 21.6±0.2° 20 using Cu Kα radiation.

28.30. (canceled)

31. The crystalline form of claim 21, wherein AMG 176 Calcium Hydrate Form 3 is further characterized by XRPD pattern peaks at 17.4, 18.6, and 20.0±0.2° 2θ using Cu Kα radiation.

32-36. (canceled)

37. The cryctallinc amorphous form of claim 17, wherein the AMG 176 Amorphous Calcium Hydrate has an endothermic transition at 292° C.±3° C., as measured by differential scanning calorimetry.

38. (canceled)

39. A pharmaceutical formulation comprising the crystalline form of claim 1 and a pharmaceutically acceptable excipient.

40. A method of treating a subject suffering from cancer, comprising administering to the subject a therapeutically effective amount of the crystalline form of claim 1.

41. The method of claim 40, wherein the cancer is multiple myeloma, non-Hodgkin's lymphoma, or acute myeloid leukemia.
